(19) United States
(12) Patent Application Publication
(10) Pub. No.: US 2023/0233822 A1
(43) Pub. Date: Jul. 27, 2023

(54) METHODS AND DEVICES FOR TREATMENT OF NEUROPATHY

(71) Applicant: University of Maine System Board of Trustees, Orono, ME (US)

(72) Inventors: Kristy Lynn Townsend, Bangor, ME (US); Rosemary Lillian Smith, Bangor, ME (US); Magdalena Blaszkiewicz, Orono, ME (US)

(21) Appl. No.: 17/923,293

(22) PCT Filed: May 7, 2021
(86) PCT No.: PCT/US2021/031337

§ 371 Date: Nov. 4, 2022

Related U.S. Application Data
(60) Provisional application No. 63/022,258, filed on May 8, 2020.

(51) Int. Cl. A61M 37/00; A61N 1/04

(52) U.S. Cl. CPC A61M 37/0015; A61N 1/04; A61M 2037/0023

(57) ABSTRACT

Disclosed herein are methods, devices, and systems for evaluation, diagnosis, prevention, and treatment of peripheral neuropathies.

### CROSS REFERENCE TO RELATED APPLICATIONS

The present application claims priority to U.S. Provisional Application No. 63/022,258, filed May 8, 2020, the entire contents of which are hereby incorporated by reference in its entirety.

### BACKGROUND

Peripheral Neuropathy (PN) refers to a condition that results when nerves that carry messages to and from the brain and spinal cord from and to the rest of the body are damaged or diseased. An estimated 30 million Americans suffer from this painful and debilitating disease, which is marked by a progressive dying back of distal nerves that starts in the skin and moves inwards, causing a complex suite of symptoms that include pain, loss of sensation, numbness, and even limb amputation. These drastic clinical outcomes are often due to subpar diagnostic tools that cannot provide early or sufficiently sensitive diagnosis of neuropathy. For certain peripheral neuropathies, early and/or more sensitive/functional diagnosis can enable interventions to prevent further decline.

Currently there is no cure for PN, and treatment is largely palliative (for example, analgesics for pain relief). However, early detection and monitoring affords the possibility of interventions to halt the progression of neuropathy, and potentially reverse the disease with novel treatments that are currently under research. As new therapies are available and in use, monitoring nerve recovery and regrowth is also a clinical need. Thus, there exists a need for improved diagnostic systems and methods for early diagnosis, monitoring, prevention, and treatment of PN.

### SUMMARY

Presented herein are systems, methods, and devices related to biomedical devices that can provide, inter alia, early-onset, sensitive, and/or functional detection of neuropathy with the goal to better control the disease. Early detection of neuropathy during clinical progression (the disease is degenerative with time) enables clinicians to mitigate the causes of neuropathy when known (for example, glucose regulation for diabetes, discontinuation or switching chemotherapy medications, improvement of environment that may include neuropathic chemicals, treatment of autoimmune diseases, etc.). In some embodiments, systems and methods described herein allow for rapid or faster detection of neuropathy that provides clinicians with quicker diagnosis times that can enable interventions to prevent further decline. In some embodiments, systems as described herein, may be utilized as ‘theragnostic’ (therapeutic and diagnostic) systems, for example, that not only provide accurate, early, and/or functional detection of neuropathy especially for small, free nerve fibers, but also allow delivery of treatments (e.g. therapeutic agents, stimulation) for prevention and treatment of the disease (e.g., via transdermal delivery, for example, through at least some needles of arrays as described herein). Furthermore, in some embodiments, devices as described herein allow for delivery of treatments, for example, to stimulate nerve re-growth and/or control pain. Other treatments targeted to skin and underlying tissue layers reached by needles of the arrays described herein, could also be delivered for treating other or related medical conditions in the same patients. In accordance with various embodiments, provided systems may allow for relatively pain-free and non-invasive testing, diagnosis, and even treatment of particular diseases, disorders or conditions. In some embodiments, methods, devices, and systems described herein facilitate biological sample collection, delivery of treatments, for example therapeutic agents and nerve stimulation, and nerve re-growth monitoring. In some embodiments, methods, systems, and/or devices as described herein may be used for monitoring nerve re-growth following a medical intervention (e.g., following a nerve graft, administration of a therapeutic agent, or following nerve stimulation) and/or the progression and/or regression of symptoms corresponding to a disease, disorder, or condition of interest. In the context of neuropathy, the potential for such diagnosis and/or intervention may improve the quality of life for millions of patients, thus saving on healthcare costs for pain medications, neuropathy related injuries, and limb amputations. Improved detection and diagnosis also allows clinicians to save time and money in patient interactions and testing.

Among other things, systems, devices, and methods, for example, as described herein, identify and address at least some of the challenges of early and accurate detection and treatment of various diseases, disorders, or conditions disclosed herein, for example, neuropathies. In some embodiments, provided systems and methods are portable, easy to use, wireless, and able to be administered in a doctor's office, clinic or eventually at home, and would fill a gap in the current market, since an accurate, specific, non-invasive, painless, easy to deliver, early onset neuropathy diagnostic and treatment system currently does not exist. It is also beneficial for the clinician to remove the subjective patient self-reporting, as is common with other neuropathy tests, by providing a functional and quantitative unbiased assessment of nerve integrity. This testing platform further improves clinical testing by being portable, inexpensive, disposable (e.g. arrays only), easy to operate, and quick (e.g. testing time of 5-10 min on average).

In one aspect, the present disclosure provides methods of evaluating and/or treating a subject for neuropathy, and/or evaluating nerve health, nerve function, and/or nerve regrowth in a subject. In some embodiments, such methods may comprise: placing a device (for example, as disclosed herein) on or below a surface of the skin of the subject, inserting one or more needles of the device into the skin and/or subcutaneous tissue of the subject, and performing one or more of an electrical measurement, a stimulation (e.g. mechanical stimuli, thermal stimuli, etc.), fluid sampling, and administration of one or more therapeutic agents using at least one of the one or more inserted needles, over a period of time, to evaluate and/or treat a subject. In some embodiments, a method further comprises determining, by comparing a performed electrical measurement with a similar electrical control measurement, a likelihood that the subject suffers from neuropathy. In some embodiments, an electrical control measurement is obtained from healthy control subjects, a patient data sample taken earlier in disease progression, or from a healthy region of tissue on the subject's body.

In some embodiments, the step of performing comprises performing two or more of an electrical measurement, a stimulation, fluid sampling, and administration of agents. In some embodiments, for example, this step is performed using at least one of the one or more inserted needles, over a period of time, to evaluate and/or treat a subject. In some embodiments, performing an electrical measurement comprises obtaining a measure of nerve conductance and/or tissue electrical activity, and/or local field potentials (e.g. extracellular recordings). In some embodiments, for example, the one or more inserted needles is recording from one section of its length (e.g. the needle's interior or exterior). In some embodiments, the one or more inserted needles is recording from its tip (e.g. if coated with insulating material). In some embodiments, the one or more inserted needles is recording from the entire length of the needle(s). In some embodiments, the needles may be at any distance from each other in the array in order to provide a discernable electrical signal.

In some embodiments, the step of performing an electrical measurement over the period of time comprises: measuring electrical potential (or current) via at least one of the inserted needles over the period of time, wherein the electrical potential (or current) is measured versus a reference electrode; and determining a measure of nerve conductance associated with the measured electrical potential (or current). In some embodiments, performing an electrical measurement comprises: at each of a plurality of target depths within the skin or subcutaneous tissue, measuring electrical potential (or current) of at least one inserted needle over a period of time; and determining, for each of the plurality of target depths, a measure of nerve conductance associated with the measured electrical potential (or current) for the target depth. In some embodiments, a plurality of target depths comprise two or more depths within the skin and/or subcutaneous tissue associated with neuropathy.

In some embodiments, a measure of nerve conductance is determined based on a characteristic frequency of potential (or current) maxima measured in a given period of time. In some embodiments, a measure of nerve conductance is determined based on a characteristic amplitude of current measured in a given period of time. In some embodiments, a measure of nerve conductance is determined based on a characteristic voltage associated with potential (or current) maxima measured in a given period of time. In some embodiments, a measure of nerve conductance is determined based on a characteristic voltage level of a measured electrical potential (or current) in a given period of time. In some embodiments, a measure of nerve conductance is determined based on an average of measured electrical potentials (or currents) acquired from each inserted needle of the array. In some embodiments, a measure of nerve conductance is determined based on a characteristic frequency of potential (or current) minima measured in a given period of time. In some embodiments, a measure of nerve conductance is determined based on a characteristic voltage associated with potential (or current) minima measured in a given period of time. In some embodiments, a measure of nerve conductance is determined based on the rate of voltage or current pulses (e.g. in a given period of time). In some embodiments, a measure of nerve conductance is determined based on amplitude of voltage or current pulses (e.g. in a given period of time). In some embodiments, a measure of nerve conductance at each of a plurality of target depths comprises a measurement of nerve die-back as a function of distance from a skin surface inward. In some embodiments, a measurement of nerve die-back provides a measure of delivery of one or more therapeutics to one or more of the plurality of target depths. In some embodiments, a measure of nerve conductance is determined by a train of electrical spikes (e.g. a spike train) that fires in a row over a given period of time (e.g. a temporal signature). In some embodiments, a measure of nerve conductance is determined by the duration between consecutive electrical spikes (e.g. a spike train) that fires in a row over a given period of time (e.g. time period of a temporal signature). In some embodiments, a measure of nerve conductance is determined by the average duration between consecutive electrical spikes (e.g. a spike train) that fires in a row over a given period of time (e.g. average time period of a temporal signature). In some embodiments, a measure of nerve conductance is determined by bursts of electrical activity (e.g. electrical spikes, or change in voltage, current, frequency, etc.) over a given period of time. In some embodiments, a measure if nerve function is determined by a measurement of the local field potential (LFP) over a given period of time.

In some embodiments, the step of determining a measure of nerve conductance comprises: determining a characteristic frequency of potential (or current) maxima measured in a given period of time; and comparing the characteristic frequency to a pre-determined threshold frequency to determine a measure of nerve conductance. In some embodiments, a pre-determined threshold frequency is obtained from a healthy subject. In some embodiments, the step of determining the measure of nerve conductance comprises: determining a characteristic value associated with potential (or current) maxima measured in a given period of time; and comparing the characteristic value to a pre-determined threshold value in order to determine a measure of nerve conductance. In some embodiments, a characteristic value is an average of one or more maxima of measured electrical potential (or current) during a given period of time. In some embodiments, the step of determining a measure of nerve conductance comprises: determining a characteristic voltage (or current) level of measured electrical potential (or current) in a given period of time; and comparing the characteristic voltage (or current) level to a pre-determined threshold value in order to determine a measure of nerve conductance. In some embodiments, the step of determining a measure of nerve conductance comprises: determining a characteristic amplitude of the measured electrical potential (or current) in a given period of time; and comparing the characteristic amplitude to a pre-determined threshold value in order to determine a measure of nerve conductance. In some embodiments, the step of determining a measure of nerve conductance comprises determining a characteristic shape of action potentials, duration of compound action potentials, excitatory potentials, or waves of electrical activity at the measured electrical potential (or current) in a given period of time. In some embodiments, a pre-determined threshold value is obtained from a healthy subject. In some embodiments, the step of determining a measure of nerve conductance comprises: determining a characteristic pattern of firing of neurons at the measured electrical potential (or current) in a given period of time; and comparing the characteristic pattern of firing of neurons to a pre-determined threshold pattern in order to determine a measure of nerve conductance. In some embodiments, a pre-determined threshold pattern is obtained from a healthy subject. In some embodiments, the step of determining a measure of nerve conductance comprises determining the tissue depth at which electrical activity is recorded.

In some embodiments, the step of performing comprises: sampling interstitial fluid of a subject from the skin and/or subcutaneous tissue using one or more inserted needles; and determining, based at least in part on properties of the sampled interstitial fluid, the status of disease progression in the subject.

In some embodiments, the step of performing comprises administering one or more therapeutic agents using at least one of the one or more inserted needles, over a period of time, to evaluate and/or treat a subject. In some embodiments, administering one or more therapeutic agents to a subject reverses and/or prevents progression of disease. In some embodiments, administering one or more therapeutic agents to a subject comprises providing the one or more therapeutic agents to the skin and/or subcutaneous tissue of the subject.

In some embodiments, one or more therapeutic agents is selected from the group consisting of a pharmacological inhibitor, a growth factor, a gene therapy agent, a drug, a biological, or a combination thereof.

In some embodiments, the step of performing comprises stimulating (e.g., one or more nerves) using at least one of the one or more inserted needles, over a period of time, to evaluate and/or treat a subject. In some embodiments, stimulation is achieved by electrically stimulating skin or subcutaneous tissue of a subject to prevent and/or reverse progression of disease. In some embodiments, stimulation through temperature regulation comprises heating or cooling skin and/or subcutaneous tissue of a subject. In some embodiments, cooling is performed using ice, a thermoelectric cooler, or the like. In some embodiments, heating is performed using heating pad or the like. In some embodiments, stimulation is achieved by mechanically stimulating skin and/or subcutaneous tissue of a subject. In some embodiments, the step of stimulating is performed using a means to provide mechanical vibration thereby mechanically stimulating muscle and/or other tissues near one or more inserted needles to stimulate nerve re-growth.

In another aspect, the present disclosure provides a system for evaluating, diagnosing, preventing, and/or treating neuropathy in a subject, comprising: a device, a means for providing fluid flow, and one or more processors (e.g. digital processors) and associated electronics configured to receive data from the device and to control the device. In some embodiments, one or more processors (e.g. digital processors) and associated electronics are configured to filter/process data received from the device. In some embodiments, a system as disclosed herein further comprises a battery to power the system. In some embodiments, a system further comprises a wireless transmission module to wirelessly exchange data to and from an operating device, for example, by Wi-Fi and/or Bluetooth. In some embodiments, a system further comprises a PC-card and/or an amplifier.

In some embodiments, a device as disclosed herein may comprise a means for providing fluid flow. In some embodiments, a means for providing fluid flow is or comprises a mechanical pump and/or a syringe pump. For example, in some embodiments, a means for providing fluid flow is fluidically coupled to at least one hollow needle of the device.

In some embodiments, a processor of a system disclosed herein is configured to perform any one or more methods disclosed herein. In some embodiments, a processor is configured to, responsive to a determination by the processor that a subject suffers from neuropathy, automatically provide a fluid to the skin and/or subcutaneous tissue of a subject via a hollow needle of the device. In some embodiments, a fluid comprises one or more therapeutic agents.

In another aspect, the present disclosure provides a device comprising: a substrate and an array of needles disposed on or through the substrate. In some embodiments, one or more needles of the array is hollow through the entire length of the needle. In some embodiments, an array as disclosed herein is configured to allow fluid transport through at least one hollow needle of the array. In some embodiments, an array as disclosed herein is configured to receive and communicate one or more electrical signals. In some embodiments, at least one of the needles of the array (e.g., an inserted needle) is capable of transmitting electrical signals to and/or from a subject (e.g. penetrated tissue of a subject).

In some embodiments, least two or more needles are of different length. In some embodiments, a length of the needles between 10 μm to 12 mm. In some embodiments, one or more needles of the array are microneedles. In some embodiments, an outer diameter of the needles is 100 μm or greater. In some embodiments, an inner diameter of the needles is 10 μm or greater. In some embodiments, a length of one or more needles is configured to allow electrical measurement. In some embodiments, a length of one or more needles is configured to allow therapeutic delivery to a target depth within the skin and/or subcutaneous tissue of a subject.

In some embodiments, a substrate and/or array, as disclosed herein, are configured to allow adjustment of depth of penetration of one or more needles into skin and/or subcutaneous tissue of a subject, thereby allowing measurement and/or treatment at a plurality of target depths within skin, and/or subcutaneous tissue of a subject. In some embodiments, depth of penetration of one or more needles is configured using an adjustable spacer. In some embodiments, a substrate is or comprises a flexible backing. In some embodiments, a substrate is a square, rectangle, or other shape suitable to accommodate curvature of various body parts. In some embodiments, a substrate has a surface area of at least 1 square inch. In some embodiments, substrate has at least one dimension of at most 8 inches.

In some embodiments, one or more needles in an array as disclosed herein is or comprises stainless steel, silicon, platinum, gold, silver, copper, any electrically conductive metal, or any combination thereof. In some embodiments, one or more needles in the array further comprises a first coating to penetrate skin to epidermis, dermis, or further beneath the skin (e.g., such as to adipose or muscle tissues). In some embodiments, one or more needles in the array further comprises a second coating to insulate an electrical signal. In some embodiments, one or more needles in the array comprises two or more coatings. In some embodiments, one or more needles in the array is coated with an electrically conductive material. In some embodiments, one or more needles in the array is at least partially filled with an electrically conductive material. In some embodiments, an electrically conductive material is selected from the group consisting of stainless steel, silicon, platinum, gold, silver, copper, polymers, any metal, or any combination thereof. In some embodiments, one or more needles comprise an electrically conductive wire through the hollow opening. In some embodiments, one or more needles in the array is or comprises an electrically non-conductive material. In some embodiments, one or more needles is at least partially filled or coated with an electrically non-conductive material.

In some embodiments, one or more of the needles is a stimulating electrode. In some embodiments, one or more needles is a reference electrode or ground electrode.

In some embodiments, fluid transport through at least one hollow needle of the array may be bidirectional. In some embodiments, fluid transport is between a device as disclosed herein and one or more of skin, interstitial fluid, and/or subcutaneous tissue of a subject.

In some embodiments, a device as disclosed herein further comprises a heating or cooling mechanism. In some embodiments, a device as disclosed herein further comprises a mechanism of stimulation. In some embodiments, a mechanism of stimulation is electrical. In some embodiments, electrical stimulation is achieved by administration of approximately 20% greater voltage/current than required for maximal stimulus. In some embodiments, mechanism of stimulation is mechanical. In some embodiments, mechanical stimulation is achieved by vibration. In some embodiments, mechanism of stimulation is through temperature regulation.

In some embodiments, a device and/or a system as disclosed herein is 3D printed or otherwise fabricated.

### BRIEF DESCRIPTION OF THE DRAWING

The foregoing and other objects, aspects, features, and advantages of the present disclosure will become more apparent and better understood by referring to the following description taken in conjunction with the accompanying figures, in which:

FIG. 1a is a schematic of the theragnostic device/system as described herein, according to an illustrative embodiment. The schematic shows a needle array with wired I/O (input/output).

FIG. 1b is a schematic of the theragnostic device/system as described herein, according to an illustrative embodiment. The schematic shows a needle array with wireless I/O (input/output).

FIG. 2 is a schematic showing disease progression, according to an illustrative embodiment. In some embodiments, PN extends to subcutaneous adipose tissue and involves dying back of nerves from the skin inwards. PN extending beyond the skin to the underlying adipose tissue contributes to overall metabolic dysfunction due to disruption of brain-adipose communication.

FIG. 3 shows measurement of protein levels in human and mouse subcutaneous adipose tissue with (i) neuropathy with obesity (panels A and B), and (ii) neuropathy with aging (panels C and D), according to an illustrative embodiment.

FIG. 4 shows results of stainless steel needle recordings of nerve conductance and delivery of solubilized material subdermally. (A) The system as disclosed herein (i.e comprising a needle (e.g. microneedle) array) was used to record nerve conductance from mouse flank skin. The blue vertical line indicates recorded compound action potentials. (B) Individual needles delivered 2% Evan's Blue dye subdermally into the subcutaneous adipose tissue of the mouse. (C) Photograph of the dye stained inguinal subcutaneous adipose depot, demonstrating feasibility of targeted substance delivery via the recording needles, directly to an area with recorded neuropathy deficits.

FIG. 5 is a schematic of an exemplary study for testing certain embodiments in known diet-induced and genetic models of diabetic peripheral neuropathy.

FIG. 6 is a schematic of a system for use in on-site processing including signal amplification and wireless communication between a device as disclosed herein and a computing device. A. Schematic of on-site signal amplification and wired communication between DEN Module prototype and FOX-DEN software. Needle arrays with wired I/O are connected to signal processing hardware on a computer. B. Schematic of in situ signal amplification and wireless communication between DEN Module prototype and FOX-DEN software. Recordings of electrical activity are transmitted via Bluetooth or WiFi to data processing software (e.g., an application on computer or tablet).

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Agent: In general, the term “agent”, as used herein, may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Biocompatible: The term “biocompatible”, as used herein, refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects.

Biological Sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological cells. In some embodiments, a biological sample is or comprises biological tissue or fluid. In some embodiments, a biological sample may be or comprise blood; blood cells; tissue (e.g. skin) or fine needle biopsy samples; cell-containing body fluids; free floating nucleic acids; lymph; skin swabs; extracellular fluid, interstitial fluid and molecules contained therein, other body fluids (e.g. sweat), secretions, and/or excretions; and/or cells therefrom, etc. In some embodiments, a biological sample is or comprises interstitial fluid. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Biomarker: The term “biomarker” is used herein, consistent with its use in the art, to refer to an entity, event, or characteristic whose presence, level, degree, type, and/or form, correlates with a particular biological event or state of interest, so that it is considered to be a “marker” of that event or state. To give but a few examples, in some embodiments, a biomarker may be or comprise a marker for a particular disease state, or for likelihood that a particular disease, disorder or condition may develop, occur, or reoccur. In some embodiments, a biomarker may be or comprise a marker for a particular disease or therapeutic outcome, or likelihood thereof. Thus, in some embodiments, a biomarker is predictive, in some embodiments, a biomarker is prognostic, in some embodiments, a biomarker is diagnostic, of the relevant biological event or state of interest. A biomarker may be or comprise an entity of any chemical class, and may be or comprise a combination of entities. For example, in some embodiments, a biomarker may be or comprise a nucleic acid, a polypeptide, a lipid, a carbohydrate, a small molecule, an inorganic agent (e.g., a metal or ion), or a combination thereof. In some embodiments, a biomarker is a cell surface marker. In some embodiments, a biomarker is intracellular. In some embodiments, a biomarker is detected outside of cells (e.g., is secreted or is otherwise generated or present outside of cells, e.g., in a body fluid such as blood, interstitial fluid, tears, saliva, etc.). In some embodiments, a biomarker is detected in a biological sample. In some embodiments, a biomarker may be or comprise a genetic or epigenetic signature. In some embodiments, a biomarker may be or comprise a gene expression signature.

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Comprising: A composition or method described herein as “comprising” one or more named elements or steps is open-ended, meaning that the named elements or steps are essential, but other elements or steps may be added within the scope of the composition or method. It is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Electrical activity: As used herein, the term “electrical activity” refers to any characteristics (e.g. frequency, amplitude, shape, voltage, rate, pattern, temporal signature, or other) of electrical signals measured within and beneath the skin for the purpose of determining clinical diagnosis.

Electrically conductive: As used herein, the term “electrically conductive” refers to the property of allowing the flow electrical current. For example, an electrically conductive material may be a metal, metal alloy, conducting polymers or glassy carbon. In some embodiments, an electrically conductive material has a conductivity of 10⁵ siemens per meter (S/m) or greater at room temperature.

In vitro: The term “in vitro” as used herein refers to events that occur in an artificial environment, e.g., in a test tube or reaction vessel, in cell culture, etc., rather than within a multi-cellular organism.

In vivo: as used herein refers to events that occur within a multi-cellular organism, such as a human and/or a non-human animal. In the context of cell-based systems, the term may be used to refer to events that occur within a living cell in a multi-cellular organism (as opposed to, for example, in vitro systems).

Microneedle: The term “microneedle” as used herein generally refers to an elongated structure with diameter less than a millimeter that is of suitable length and shape to penetrate skin. In some embodiments, a microneedle is arranged and constructed (by itself or within a device) to provide the ability to electrically communicate with nerves when inserted into skin, while still creating efficient pathways for drug delivery. In some embodiments, a microneedle has a diameter which is consistent along the microneedle's length. In some embodiments, a microneedle has a diameter that changes along the microneedle's length. In some embodiments, a microneedle has a diameter that tapers along the microneedle's length. In some embodiments, a microneedle's diameter is narrowest at the tip that penetrates skin. In some embodiments, a microneedle may be solid. In some embodiments, a microneedle may be hollow. In some embodiments a microneedle may be tubular. In some embodiments, a microneedle may be sealed on one end. In some embodiments, a plurality of microneedles is utilized. In some embodiments, a plurality of microneedles is utilized in an array format. In some embodiments, a microneedle may have a length within a range of about 1 μm to about 24,000 μm. In some embodiments, a microneedle may have a length of at least about 50 μm.

‘Neuropathy’ or ‘Peripheral neuropathy’: As used herein, the terms “neuropathy” and “peripheral neuropathy” refer generally to dysfunction, for example caused by damage or disease affecting nerves (e.g., peripheral nerves). Neuropathy may be associated with a decrease in nerve conductance or aberrations in nerve electrical activity, in an affected portion of the body. Neuropathy may be idiopathic (i.e. no known cause) or caused by, for example, a disease (e.g., diabetes), physical trauma, genetic background, and/or an infection.

Prevention: The term “prevention”, as used herein, refers to a delay of onset, and/or reduction in frequency and/or severity of one or more symptoms of a particular disease, disorder or condition. In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition. Prevention may be considered complete when onset of a disease, disorder or condition has been delayed for a predefined period of time.

Risk: as will be understood from context, “risk” of a disease, disorder, and/or condition refers to a likelihood that a particular individual will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, relative risk is 0, 1, 2, 3, 4, 5, 6, 7, 8, 9, 10, or more.

Subject: As used herein, the term “subject” refers an organism, typically a mammal (e.g., a human). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutic agent: As used herein, the phrase “therapeutic agent” in general refers to any agent that elicits a desired pharmacological or clinical effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutic regimen: A “therapeutic regimen”, as that term is used herein, refers to a dosing regimen whose administration across a relevant population may be correlated with a desired or beneficial therapeutic outcome.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. In some embodiments, treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

### DETAILED DESCRIPTION

Among other things, the present disclosure describes systems and methods for early detection, prevention, and/or treatment of certain disorders or conditions, for example, associated with the nervous system (e.g., conditions associated with neuropathies) through the use of a novel biomedical system that, inter alia, can detect and monitor nerve signals (e.g. nerve conductance). In accordance with various embodiments, provided systems and methods may also include the ability to sample a subject's local environment (e.g., tissue or body fluids), and/or, administer one or more treatments (e.g., novel therapeutic agents and/or nerve stimulation) to potentially delay and/or prevent progression, or treat a disease, disorder, or condition. In some embodiments, the present disclosure provides methods for treating neuropathies. In some embodiments, the present disclosure provides treatments for one or more of diabetic peripheral neuropathy (DPN), chemotherapy-induced neuropathy (CIPN), HIV or AIDS-induced neuropathy; and/or idiopathic peripheral neuropathies (IPN), neuropathies with no identifiable known cause (such as with aging). In some embodiments, the present disclosure provides systems and methods for diagnosis, prevention, and treatment for DPN.

In some embodiments, provided systems and methods as described herein, can not only record electrical signals (e.g. local field potentials, nerve conductance, etc. as described herein) but also sample biological fluids, for example, for biomarker analysis, and even administer one or more treatments such as one or more therapeutic agents and/or one or more forms of stimulation in a non-invasive (or minimally invasive) manner. Delivery of substances may also be utilized to stimulate nerve activity prior to or during electrical measurements (for example, delivery of capsaicin to stimulate skin and subcutaneous adipose sensory nerves expressing the cation channel TRPV1). In some embodiments, an array of needles is configured to allow electrical measurement and/or sampling of a subject's bodily fluid(s) within an appropriate surface area for a given application. In some embodiments, an array of needles is configured to allow electrical measurement and/or delivery of treatment(s) within an appropriate surface area for a given application. In some embodiments, systems and methods as described herein are used to record electrical signals (e.g. voltage, current, local field potentials, nerve conductance, etc. as described herein) to calculate a measure of nerve conductance of nerves under the surface of skin. In some embodiments, a subset of an array of needles may be used to record and measure electrical signals, while a different or even the same subset of the array of needles is configured to sample a subject's bodily fluid(s) and/or administer treatments.

In some embodiments, treatments may be or comprise therapeutic agents. In some embodiments, therapeutics agents may be or comprise drug therapies. In some embodiments, treatments may be or comprise nerve stimulation (e.g. to initiate re-growth of dying nerves), for example via temperature-based (e.g. hot versus cold) stimulation, or mechanical (e.g. vibration) stimulation.

Systems and methods disclosed herein, may be used to obtain nerve recordings from varying depths from the surface of the skin. In some embodiments, needles may be used to obtain nerve recordings (i.e. electrical measurements) In some embodiments, length of the needles and depth of penetration dictate the depth at which nerve recordings may be obtained. In some embodiments, nerve recordings may be obtained from a subject, either at the surface of the skin, or in one or more sublayers beneath the skin's surface. Moreover, in some embodiments, needles may be used to obtain biological samples from a subject, which may be used for biomarker analysis. Thus, systems and methods disclosed herein enable early diagnosis and interventions for disclosed diseases, disorders, or conditions, and provide a promise to improve the quality of life for millions of patients.

In some embodiments, detection and diagnosis of a disease, disorder, or condition, recording of nerve signals (i.e. electrical signals such as voltage, conductance, etc. as disclosed herein), and administration of treatment is achieved through interaction with and/or penetration of disclosed systems with one or more components of the skin (e.g., transdermally). This allows for minimally invasive and painless detection, recording, sampling, and treatment of various diseases, disorders, and conditions, a functionality that currently not available to patients. For example, currently, diagnosis of neuropathies is performed by highly invasive and time-consuming tests (e.g. nerve function tests, biopsies, etc.) that detect late-stage neuropathies causing extreme discomfort to patients and disrupting their lives. Furthermore, there exists no treatment for neuropathy, let alone late-stage neuropathy, and treatment goals typically are to manage symptoms including pain. Thus, there exists a need for a system that not only accurately detects neuropathy (e.g. PN) early on, but also provides a way to manage its progression, and administer therapies as and when required in order to prevent and treat (e.g. stimulate nerve regrowth) in neuropathy patients.

In some embodiments, provided systems and methods as described herein, can be used for the monitoring of nerve regrowth.

In some embodiments, provided systems and methods as described herein, can be used as a means of clinical screening for small fiber peripheral neuropathy, or other nerve dysfunction.

### I. METHODS OF DIAGNOSIS, PREVENTION, AND TREATMENT

Methods for diagnosis, sampling, prevention, and treatment of various diseases, disorders, or conditions, are described herein. In some embodiments, methods disclosed herein are used to diagnose, sample, prevent, and treat neuropathies. In some embodiments, methods disclosed herein are used to diagnose, sample, prevent, and treat PN. In some embodiments, methods disclosed herein are used to diagnose, sample, prevent, and treat other conditions affecting skin and/or subdermal tissues (e.g. peripheral arterial disease, lymphatic dysfunction, fibromyalgia, etc.).

### Diagnosis/Detection:

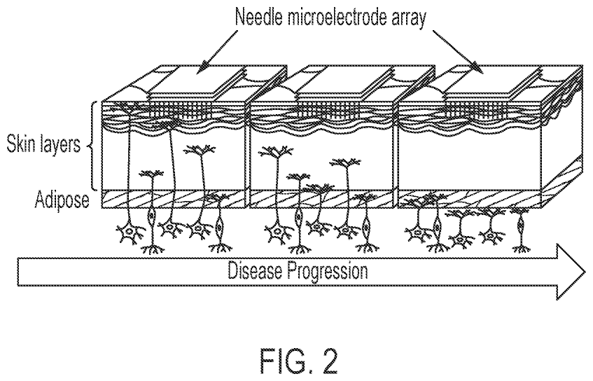

Systems disclosed herein may be used for early onset detection of various diseases, disorders, and/or conditions as disclosed herein. In some embodiments, systems disclosed herein may be used for detection and/or diagnosis of neuropathies, for example, peripheral neuropathy. In some embodiments, provided systems may be used to measure nerve conductance in small nerve fibers of the skin and underlying tissues, allowing for sensitive and early diagnosis of neuropathy by detecting loss of nerve signal due to neurodegeneration. In some embodiments, provided systems may be used to measure nerve conductance of one or more types of nerve fibers (e.g. peripheral nerve fibers, sensory nerve fibers, motor nerve fibers, autonomic nerve fibers, and associated nerve fiber subtypes (i.e. group A, B, and C fibers)) of the skin and underlying tissues and obtaining an average of the measure of nerve conductance from penetrated tissue (e.g. penetrated with one or more needles of needle array). In some embodiments, provided systems may be used to measure nerve conductance of two of more types of nerve fibers (e.g. peripheral nerve fibers, sensory nerve fibers, motor nerve fibers, autonomic nerve fibers, and associated nerve fiber subtypes (i.e. group A, B, and C fibers)) of the skin and underlying tissues and obtaining an average of the measure of nerve conductance from penetrated tissue (e.g. penetrated with one or more needles of needle array). In some embodiments, provided systems may be used to measure nerve conductance of multiple (e.g. one or more, two or more, three or more, any and all etc.) types of nerve fibers (e.g. peripheral nerve fibers, sensory nerve fibers, motor nerve fibers, autonomic nerve fibers, and associated nerve fiber subtypes (i.e. group A, B, and C fibers)) of the skin and underlying tissues and obtaining an average of the measure of nerve conductance from penetrated tissue (e.g. penetrated with one or more needles of needle array). In some embodiments, provided systems may be used to measure nerve conductance of all types of nerve fibers (e.g. peripheral nerve fibers, sensory nerve fibers, motor nerve fibers, autonomic nerve fibers, and associated nerve fiber subtypes (i.e. group A, B, and C fibers)) of the skin and underlying tissues and obtaining an average of the measure of nerve conductance from penetrated tissue (e.g. penetrated with one or more needles of needle array). FIG. 2 is a schematic showing neuropathic disease progression and use of a theragnostic system to diagnose/detect (e.g. monitor disease, record signals (e.g. nerve conductance, impedance, voltage, current, etc.), etc.) and/or treat neuropathic disease progression according to an illustrative embodiment.

In one aspect, a method of diagnosing and/or evaluating a subject potentially at risk for or suffering from neuropathy comprises: placing a system as disclosed herein on the surface of skin of a subject, with needles protruding below the skin surface. In some embodiments, for example, a system as disclosed herein comprises a substrate and an array of needles disposed on or through the substrate, wherein one or more of the needles is hollow through the entire length of the needle, the array is configured to allow fluid transport through at least one hollow needle of the array, and the array is configured to receive and communicate one or more electrical signals. In some embodiments, systems disclosed herein are placed on skin surface of a subject. In some embodiments, systems disclosed herein are placed below skin surface of a subject.

Methods of diagnosing and/or evaluating a subject further comprises inserting one or more needles of an array into a subject's skin. In some embodiments, one or more needles are inserted into epidermis of a subject. In some embodiments, one or more needles are inserted into one or more layers of epidermis of a subject, for example, into one or more of the stratum corneum, stratum lucidum, stratum granulosum, stratum spinosum, and/or stratum basale. In some embodiments, one or more needles are inserted into dermis of a subject. In some embodiments, one or more needles are inserted into hypodermis of a subject. In some embodiments, one or more needles are inserted into subcutaneous tissue of a subject.

One or more needles of systems disclosed herein may be inserted to one or more desired target depths into or under the skin of a subject. In some embodiments, target depths may range between 1 μm to 12 mm. In some embodiments, target depths may range between 10 μm and 10 mm. In some embodiments, target depths may range between 100 μm and 5 mm. In some embodiments, target depths may range between 150 μm and 2 mm. In some embodiments, a target depth may be at least 10 μm. In some embodiments, a target depth may be at least 50 μm. In some embodiments, a target depth may be at least 100 μm. In some embodiments, a target depth may be at least 200 μm. In some embodiments, a target depth may be at least 300 μm. In some embodiments, a target depth may be at least 400 μm. In some embodiments, a target depth may be at least 500 μm. In some embodiments, a target depth may be at least 1000 μm. In some embodiments, a target depth may be at least 2000 μm. In some embodiments, a target depth may be at most 12000 μm. In some embodiments, a target depth may be at most 24000 μm. In some embodiments, for example, a substrate and/or needle array of systems disclosed herein are configured to allow adjustment of the depth of penetration (i.e. target depth) of one or more needles into skin and/or subcutaneous tissue of a subject. This allows for detection (e.g. through signal measurement), sampling, and/or delivery of treatment at a plurality of target depths within the skin and/or subcutaneous tissue of a subject. For example, the depth of penetration of one or more needles of an array may be configured, in some embodiments, using a spacer of adjustable or varying thickness, through which the needle are inserted prior to penetration of the skin. In some embodiments, needle length may be used to configure and/or control depth of penetration under skin.

In some embodiments, methods disclosed herein comprise measuring signals at each of a plurality of target depths within skin. In some embodiments, a substrate and/or array are configured to allow adjustment of the depth of penetration of one or more needles into skin and/or subcutaneous tissue of a subject, thereby allowing measurement at a plurality of target depths within the skin of a subject. In some embodiments, signals may be measured at 2 or more different target depths. In some embodiments, signals may be measured at 2, 3, 4, 5, 6, 7, 8, 9, 10, or more different target depths. In some embodiments, signals may be measured at one target depth at an instance in time. In some embodiments, signals may be measured at two or more target depths at an instance in time.

In some embodiments, methods of diagnosing and/or evaluating a subject may further comprise measuring signals from penetrated tissue. In some embodiments, at least one of the inserted needles is capable of measuring and/or recording signals from the penetrated tissue. In some embodiments, measuring and/or recording signals from nerve fibers located in or near penetrated tissue is performed. In some embodiments, measuring and/or recording signals from a specific type of nerve fiber (e.g. peripheral nerve fibers, sensory nerve fibers, motor nerve fibers, autonomic nerve fibers, and associated nerve fiber subtypes (i.e. group A, B, and C fibers)) located in or near penetrated tissue is performed. In some embodiments, measuring and/or recording signals from a multiple types (e.g. 2 or more, 3 or more, etc.) of nerve fibers (e.g. peripheral nerve fibers, sensory nerve fibers, motor nerve fibers, autonomic nerve fibers, and associated nerve fiber subtypes (i.e. group A, B, and C fibers)) located in or near penetrated tissue is performed. In some embodiments, measuring and/or recording signals from all types of nerve fibers (e.g. peripheral nerve fibers, sensory nerve fibers, motor nerve fibers, autonomic nerve fibers, and associated nerve fiber subtypes (i.e. group A, B, and C fibers)) located in or near penetrated tissue is performed. In some embodiments, a signal measured is an electrical signal. In some embodiments, an electrical signal measured is an action potential. For example, action potential measured is an action potential of a nerve. In some embodiments, an electrical signal measured is a local field potential. As is known to one of skill in the art, local field potentials are transient electrical signals generated in nervous tissue and/or other tissues by the summed and synchronous electrical activity of the individual cells in that tissue. In some embodiments, an electrical signal measured is a nerve conductance. In some embodiments, an electrical signal measured is an electrical current. In some embodiments, an electrical signal measured is an electrical voltage.

In some embodiments, methods disclosed herein comprise measuring space averages of signals across a given area of tissue. In some embodiments, signals may be measured across a given area of tissue. In some embodiments, measured area may be at least about 1 square inch. In some embodiments, measured area may be at least about 2 square inches. In some embodiments, measured area may be at least about 3 square inches. In some embodiments, measured area may be at least about 4 square inches. In some embodiments, measured area may be at least about 5 square inches. In some embodiments, measured area may be at least about 10 square inches. In some embodiments, measured area may be less than 1 square inch. In some embodiments, measured area may be more than 10 square inches. In some embodiments, measured signals may be averaged over a measured area. This allows for various skin surface area to be measured, as neuropathy progresses from hands/feet further up the arms and legs, and potentially to the torso.

In some embodiments, each electrical measurement (e.g. measurement of a signal (e.g. electrical signal)) may be performed over a period of time. That is, in some embodiments, each signal measurement may be performed as a function of time. For example, in some embodiments, a signal measurement may be performed over a period of between 30 seconds to 24 hours. In some embodiments, a signal measurement may be performed over a period of between 1 minute and 10 hours. In some embodiments, a signal measurement may be performed over a period of between 2 minutes and 1 hour. In some embodiments, a signal measurement may be performed for at least 1 minute. In some embodiments, a signal measurement may be performed for at least 2 minutes. In some embodiments, a signal measurement may be performed for at least 30 minutes. In some embodiments, a signal measurement may be performed for at least 1 hour. In some embodiments, a signal measurement may be performed for at least 2 hours. In some embodiments, a signal measurement may be performed for at least 3 hours. In some embodiments, a signal measurement may be are performed for at least 5 hours. In some embodiments, a signal measurement may be performed for at least 6 hours. In some embodiments, a signal measurement may be performed for at least 12 hours. In some embodiments, a signal measurement may be performed for at most 24 hours. In some embodiments, measured signals may be averaged over time.

In some embodiments, each electrical measurement (e.g. measurement of a signal (e.g. electrical signal)) may be performed over a period of time. That is, in some embodiments, each signal measurement may be performed as a function of time. For example, in some embodiments, a signal measurement may be performed over a period of between 1 day to 1 year. In some embodiments, a signal measurement may be performed for at least 1 day. In some embodiments, a signal measurement may be performed for at least 2, 3, 4, 5, 6, or 7 days. In some embodiments, a signal measurement may be performed for at least 2, 3, or 4 weeks. In some embodiments, a signal measurement may be performed for at least 1 month. In some embodiments, a signal measurement may be performed for at least 2, 3, 4, 5, 6, 7, 8, 9, 10, 11, or 12 months or more. In some embodiments, a signal measurement may be performed for at least 1 year or more. In some embodiments, measured signals may be averaged over time.

In some embodiments, for example, signals measured may be electrical signals. In some embodiments, electrical signals may be measured as a function of frequency. For example, electrical signals may be measured in a frequency range of 1 Hz to 10 MHz. In some embodiments, electrical signals may be measured in a frequency range of 100 Hz to 5 MHz. In some embodiments, electrical signals may be measured in a frequency range of 200 Hz to 2 MHz. In some embodiments, electrical signals may be measured in a frequency range of 250 Hz to 1 MHz.

In some embodiments, electrical signals may be measured as a function of voltage. For example, electrical signals may be measured in a voltage range of 1 fV to 1 V. In some embodiments, electrical signals may be measured in a voltage range of 10 pV to 0.5 V. In some embodiments, electrical signals may be measured in a voltage range of 20 pV to 0.2 V. In some embodiments, electrical signals may be measured in a voltage range of 25 pV to 0.1 V.

In some embodiments, measurement of an electrical signal is performed using needles that serve as electrodes. In some embodiments, measurement of an electrical signal may be performed using a 2-electrode system. In some embodiments, measurement of an electrical signal may be performed using a 3-electrode system. In some embodiments, an electrical signal is measured against one or more reference electrodes. In some embodiments, one or more needles of an array serves as a reference electrode. Reference electrodes may comprise any application-appropriate materials (e.g., metals or polymers). For example, in some embodiments, reference electrodes may be biocompatible and used in combination with systems and methods disclosed herein. In some embodiments, reference electrodes may be aqueous reference electrodes. In some embodiments, reference electrodes may be non-aqueous reference electrodes. By way of additional example, in some embodiments, reference electrodes may be or comprise Ag—AgCl (Silver-silver chloride) electrodes, standard hydrogen electrodes, normal hydrogen electrodes, reversible hydrogen electrodes, saturated calomel electrodes, copper-copper(II) electrodes, palladium-hydrogen electrodes, dynamic hydrogen electrodes, mercury-mercurous sulfate electrodes, or any combination thereof. In some embodiments, one or more needles of an array may serve as a ground electrode.

In some embodiments, methods disclosed herein comprise processing measured electrical signals using a processor. In some embodiments, measured electrical signals are converted (e.g., by data processing) into an assessment of nerve conductance. In some embodiments, nerve conductance is determined as a function of frequency. In some embodiments, nerve conductance is determined for a characteristic frequency. In some embodiments, nerve conductance is determined as a function of voltage. In some embodiments, nerve conductance is determined for a characteristic voltage. In some embodiments, nerve conductance is determined for an average of the measured electrical signals acquired from each inserted needle of the array.

In some embodiments, determining an assessment of nerve conductance from electrical signals comprises determining a characteristic parameter of electrical signals and comparing a value associated with the characteristic parameter to a value of a pre-determined threshold parameter (e.g., a reference level or reference value that is comparable to the characteristic parameter). In some embodiments, a value of a pre-determined threshold parameter may be obtained from one or more healthy subjects. In some embodiments, value of a pre-determined threshold parameter may be obtained from a population of healthy subjects (e.g., an average reading (e.g., average electrical signal measurement taken from each of the members of the population)). In some embodiments, a value of a pre-determined threshold parameter may be obtained for one or more specific body locations. In some embodiments, a value of a pre-determined threshold parameter may be obtained for one or more specific skin depths. As is known to a person of ordinary skill in the art, when comparing a value of a characteristic parameter with a value of a pre-determined threshold parameter care must be taken that both have been obtained under comparable parameters (e.g., for a given skin depth, needle type, and/or body location).

In some embodiments, a characteristic parameter is a frequency or range of frequencies. In some embodiments, a characteristic parameter is a potential (or current) maxima. In some embodiments, a potential (or current) maxima is an average of one or more maxima of a measured electrical potential (or current). In some embodiments, a characteristic parameter is a voltage (or current) level of a measured electrical potential (or current). In some embodiments, a characteristic parameter is an amplitude of a measured electrical potential (or current). In some embodiments, a characteristic parameter is a pattern of firing of neurons at measured electrical potentials (or currents).

In some embodiments, a pre-determined threshold parameter is a frequency. In some embodiments, a pre-determined threshold parameter is a potential (or current) maxima. In some embodiments, a potential (or current) maxima is an average of one or more maxima of a measured electrical potential (or current). In some embodiments, a pre-determined threshold parameter is a voltage (or current) level of a measured electrical potential (or current). In some embodiments, a pre-determined threshold parameter is an amplitude of a measured electrical potential (or current). In some embodiments, a pre-determined threshold parameter is a pattern of firing of neurons at the measured electrical potential (or current).

Systems and methods disclosed herein may also be enabled to transmit recorded signals. Methods disclosed herein include, for example, transmitting electrical signals to and/or from penetrated tissue. In some embodiments, electrical signals are transmitted to a processor and/or a storage device. In some embodiments, electrical signals are further processed after transmission. In some embodiments, electrical signals are further processed before transmission.

In some embodiments, methods presented herein may further include a comparison of electrical signals obtained from a subject with electrical signals obtained from one or more control subjects. In some embodiments, for example, electrical signals (e.g. raw electrical signals, processed electrical signals etc.) obtained from a subject are compared to electrical signals obtained from control subjects (e.g. one or more subjects that do not suffer from a disease, disorder, or condition disclosed herein (negative control)), to diagnose a likelihood that subject suffers from a disease, disorder, or condition disclosed herein. In some embodiments, electrical signals obtained from a subject are compared to electrical signals obtained from control subjects (e.g. one or more subjects that suffer from a disease, disorder, or condition disclosed herein (positive control)).

In some embodiments, electrical signals obtained from a subject are compared with electrical signals obtained from controls (e.g. pre-determined threshold values). For example, by comparing electrical measurements obtained from a subject with comparable electrical control measurements, one can measure a likelihood that a subject suffers from a disease, disorder, or condition (e.g., neuropathies, PN). Systems disclosed herein may be used to measure reference values (i.e. pre-determined threshold values) from one or more healthy subject(s). In some embodiments, such thresholds or reference values may be used as comparators to diagnose a disease, disorder, or condition in subjects.

In some embodiments, methods of detection and/or evaluation disclosed herein may be performed repeatedly in subjects to evaluate progression of a disease, disorder, or condition in subjects over time (e.g. lifetime of a subject). In some embodiments, for example, successive measurements may be separated by at least 1 day. In some embodiments, for example, successive measurements may be separated by at least 1 week. In some embodiments, for example, successive measurements may be separated by at least 2 weeks. In some embodiments, for example, successive measurements may be separated by at least 3 weeks. In some embodiments, for example, successive measurements may be separated by at least 1 month. In some embodiments, for example, successive measurements may be separated by at least 2, 3, 4, 5, 6, 7, 8, 9, 10, 11, or 12 months. In some embodiments, for example, successive measurements may be separated by at least 1 year or more. In some embodiments, successive measurements may be performed at a specific target depth. In some embodiments, successive measurements may be performed to monitor progression from healthy tissue to neuropathic tissue in a subject. In some embodiments, successive measurements may be performed to monitor progression from neuropathic tissue to healthy tissue in a subject. In some embodiments, successive measurements may be performed to monitor nerve re-growth after treatment.

### Sampling:

Methods and systems disclosed herein may be equipped to obtain samples from subjects (e.g. healthy and/or diseased) and/or evaluate markers for various stages of a disease, disorder, or condition. In some embodiments, provided systems and methods may be configured to test and/or obtain biological samples from subjects. In some embodiments, samples tested and/or obtained may be used for diagnosis and/or biomarker discovery for various diseases, disorders, or conditions as disclosed herein. For example, needles inserted into skin of subjects for detection and/or evaluation of disease, disorder, or condition may be designed to be capable of sampling biological samples from penetrated tissue (e.g., in one or more body fluids).

Any of a variety of biological samples can be suitable for use with particular embodiments, as described below and elsewhere herein. In some embodiments, a biological sample is or comprises biological cells. In some embodiments, a biological sample is or comprises tissue. In some embodiments, a biological sample is or comprises skin. In some embodiments, a biological sample is or comprises biological fluids. In some embodiments, a biological fluid is or comprises interstitial fluid (ISF). In some embodiments, a biological fluid is or comprises blood. In some embodiments, a biological fluid is or comprises sweat. In accordance with various embodiments, a biological sample may be used for biomarker analysis for detection of a disease, disorder, or condition as disclosed herein, and/or for evaluating progression of a disease or condition.

In some embodiments, methods disclosed comprise obtaining one or more biological samples from a subject. In some embodiments, biological samples may be obtained from a specific target depth. In some embodiments, biological samples may be obtained using a one or more needles of an array. In some embodiments, biological samples may be obtained using all or substantially all of the needles of an array. In some embodiments, biological samples may be obtained using any of the needles of an array. For example, in some embodiments, biological samples may be obtained using needles in the center or substantially in the center of an array. In some embodiments, biological samples may be obtained using needles from an edge of an array.

Systems and methods disclosed herein may be used to evaluate (i.e. diagnose, record, and/or monitor) progression of a disease, disorder, or condition (e.g. neuropathy) by repeatedly sampling biological samples from a subject. Accordingly, in some embodiments, multiple biological samples may be obtained from a subject over a period of time. In some embodiments, successive biological samples may be obtained at least 1 day apart. In some embodiments, for example, successive biological samples may be obtained at least 1 week apart. In some embodiments, for example, successive biological samples may be obtained at least 2 weeks apart. In some embodiments, for example, successive biological samples may be obtained at least 3 weeks apart. In some embodiments, for example, successive biological samples may be obtained at least 1 month apart. In some embodiments, successive biological samples may be obtained at least 3 months, 6 month, 9 months, or 1 year apart. In some embodiments, successive biological samples may be obtained at a specific target depth. In some embodiments, successive biological samples may be obtained at different target depths. In some embodiments, successive biological samples may be obtained to evaluate progression from healthy tissue to neuropathic tissue in a subject. In some embodiments, successive biological samples may be obtained to evaluate progression from neuropathic tissue to healthy tissue in a subject. In some embodiments, successive biological samples may be obtained to evaluate (i.e. monitor, record, and/or diagnose) nerve re-growth after treatment.

Systems and methods disclosed herein may include, in some embodiments, the ability to analyze an obtained biological sample. In some embodiments, for example, obtained biological samples may be analyzed (i.e. detect qualitatively or quantitatively one or more specific biomarkers associated with a disease, disorder, or condition) on-chip (i.e. on the systems disclosed herein). For example, in some embodiments, obtained biological samples may be analyzed for one or more specific biomarkers while said biological sample is within one or more needles of a needle (e.g. microneedle) array. In some embodiments, obtained biological samples may be analyzed off-chip (for example, in a lab). In some embodiments, obtained biological samples may be analyzed quantitatively. In some embodiments, obtained biological samples may be analyzed qualitatively.

Obtained biological samples may be analyzed for one or more biomarkers using one or more sensing techniques. In some embodiments, for example, one or more biomarkers may be analyzed using electrochemical sensors. In some embodiments, one or more biomarkers may be analyzed using electronic sensors.

### Treatment:

In some embodiments, provided methods and systems may also be used for preventing and/or treating various diseases, disorders, and/or conditions. In some embodiments, methods and systems of the present disclosure may be used for prevention and/or treatment of neuropathy. For example, in some embodiments, a method of preventing and/or treating a subject potentially at risk for or suffering from neuropathy, comprises: placing, as discussed elsewhere herein, a device/system as disclosed herein on the surface of skin of a subject, with needles protruding below the skin surface. Methods further comprise inserting one or more needles into skin and/or subcutaneous tissue of a subject to one or more desired depths and obtaining signal measurements as discussed elsewhere herein. In some embodiments, at least one of the inserted needles is capable of transmitting signals to and/or from the penetrated tissue. As discussed elsewhere herein, a number of different signals may be measured/recorded and transmitted from at least one of the inserted needles, and in some embodiments, based on the measured signals, one or more treatments may be administered.

In some embodiments, one or more treatments may be administered using systems disclosed herein. In some embodiments, systems disclosed herein are able to stimulate nerve re-growth and/or revive nerve functionality; for example, through: delivery of a therapeutic agent (for example any therapeutic in solution including, but not limited to an AAV-based therapy, a small molecule, a biological agent, etc.), electrical stimulation (for example a system as disclosed herein comprises one or more stimulating electrodes included in the needle array comprising recording needles), or stimulation (for example, through temperature regulation, mechanical vibration, or any other known technologies). For example, in some embodiments, one or more treatments are administered using one or more needles inserted into subject. In some embodiments, one or more treatments are administered using all or substantially all of needles inserted into subject. In some embodiments, treatment is or comprises administering one or more therapeutic agents. In some embodiments, treatment is or comprises administering one or more drug therapies. In some embodiments, treatment is or comprises administering one or more AAV-based therapy. In some embodiments, treatment is or comprises administering one or more biological agents. In some embodiments, treatment is or comprises nerve stimulation. In some embodiments, nerve stimulation is or comprises temperature-based nerve stimulation. In some embodiments, nerve stimulation is or comprises cold nerve stimulation. In some embodiments, nerve stimulation is or comprises heat-based nerve stimulation. For example, the temperature of one or more inserted needles are raised or lowered to administer temperature-based nerve stimulation. In some embodiments, nerve stimulation is or comprises electrical stimulation. In some embodiments, nerve stimulation is or comprises mechanical stimulation (e.g. vibration (e.g. of needles)).

In some embodiments, treatments are administered over a period of time to prevent (e.g., delay onset or worsening) progression of disease, disorder, or condition, and/or to treat a subject. In some embodiments, electrical signal measurements are performed repeatedly in subjects to evaluate progression of disease, disorder, or condition in subjects over time. In some embodiments, methods of detection and/or evaluation disclosed herein may be performed repeatedly (i.e. successively) in subjects to evaluate progression of a disease, disorder, or condition in subjects over time. As disclosed herein, in some embodiments, successive measurements may be separated by varying time intervals. In some embodiments, these successive measurements may be used to evaluate a course of treatment. Thus, in some embodiments, measurements (e.g. of signals (e.g. electrical signals)) using systems disclosed herein are preceded by, followed by, or occur substantially coincident with, administration of one or more treatments.

In some embodiments, needles of systems disclosed herein are designed to be capable of transmitting signals to penetrated tissue. For example, in some embodiments, systems disclosed herein may apply stimulation to penetrated tissue and/or tissue components near penetrated tissue. In some embodiments, stimulation is electrical. In some embodiments, electrical stimulation (e.g. achieved by applying a particular voltage, current, etc.) is administered to diseased or damaged nerves of a subject. Such electrical stimulation may, in some embodiments, aide in preventing and/or reversing nerve death and/or promote nerve re-growth. In some embodiments, electrical stimulation is achieved by administration of a voltage and/or current that is approximately 20% greater than is required for typical nerve stimulation (e.g., a stimulus required to trigger a nerve to fire). In some embodiments, a stimulus is in the millivolt range. In some embodiments, a stimulus is less than 1 volt. In some embodiments, a stimulus is in the milliampere range. In some embodiments, a stimulus is in the microampere range. In some embodiments, a stimulus is less than 1 ampere. In some embodiments, a stimulus is administered as pulses. In some embodiments, an amplitude of one of more pulses may be in a range of 0.1-100 μA, 0.1-1000 μA, 0.01-100 μA, 0.01-1000 μA, or 0.001-1000 μA. In some embodiments, an amplitude of one of more pulses is in a range of 0.1-100 μA. In some embodiments, an amplitude of one of more pulses may be at least 0.001 μA. In some embodiments, an amplitude of one of more pulses may be at least 0.01 μA. In some embodiments, an amplitude of one of more pulses may be at least 0.1 μA. In some embodiments, an amplitude of one of more pulses may be at most 10 μA. In some embodiments, an amplitude of one of more pulses may be at most 100 μA. In some embodiments, an amplitude of one of more pulses may be at most 1000 μA. In some embodiments, at least 2 pulses may be administered. In some embodiments, at least 2 pulses may be administered. In some embodiments, at most 10 pulses may be administered. In some embodiments, at most 20 pulses may be administered. In some embodiments, at most 50 pulses may be administered. In some embodiments, a delay between pulses may be at least 1 mSec. In some embodiments, a delay between pulses may be at least 10 mSec. In some embodiments, a delay between pulses may be at least 100 mSec. In some embodiments, a delay between pulses may be at least 1 second. In some embodiments, the delay between pulses may be at most 1 second. In some embodiments, a delay between pulses may be greater than 1 second. In some embodiments, a delay between pulses may be less than 1 second.

Apart from stimulation, treatments for a disease, disorder, or condition disclosed herein (e.g., neuropathy) may include administration of one or more therapeutic agents (e.g. drug therapies, biological agents, etc.). In some embodiments, systems and methods provided herein may be used for administration of such therapeutic agents. In some embodiments, needles inserted into a subject may be used for administration of one or more therapeutic agents. In some embodiments, one or more therapeutic agents are administered transdermally. In some embodiments, one or more therapeutics agents are administered sub-dermally. In some embodiments, one or more therapeutic agents are administered subcutaneously.

Administration of treatments using systems and methods provided herein may be automated. For example, in some embodiments, delivery of one or more treatments (e.g. therapeutic agents or stimulation) may be automated. Automation, for example of administration of treatments may involve one or more processors. In some embodiments, one or more processors may be incorporated in to systems (e.g. on-device) described herein. In some embodiments, one or more processors may be located away (e.g. off-device) from systems described herein and may be connected to said systems wirelessly. In some embodiments, for example, a processor may be configured to, responsive to a determination by the processor that a subject suffers from a disease, disorder, or condition (e.g. neuropathy), automatically provide instructions to the systems disclosed herein to provide one or more treatments (e.g. one or more therapeutic agents (e.g. drug therapies, biological agents, etc.) and/or stimulation). In some embodiments, instructions received from a processor to a system may include information about one or more of treatment duration, treatment dosage, a period of time between successive treatment cycles, one or more target depths for treatment administration, and other such related parameters.

### Combination Therapies:

As may be known to a person of ordinary skill in the art, one or more signal measurements and/or one or more treatments disclosed herein may be combined or applied in combination to achieve more accurate detection and maximum treatment efficiency. For example, in some embodiments, mechanical stimulation may be combined with administration of one or more therapeutic agents. In another example, electrical stimulation may be combined with other forms of stimulation (e.g., mechanical or temperature based stimulation). In yet another embodiment, electrical signal measurements obtained over one or more target depths may be used as an indicator for initiating a specific treatment, for example, electrical stimulation.

Furthermore, any methods of recording and/or monitoring electrical measurements, and/or treatments (e.g. stimulation, therapeutic agents such as drug therapies, biological agents, etc.) disclosed herein may be combined with existing traditional treatments for a disease, disorder, or condition. For example, existing treatments (e.g., injections for increasing nerve pressure, pain relievers, anti-depressants, anti-seizure medications, other medications (topical and/or oral), etc.) for a disease, disorder, or condition (e.g. neuropathy) may be combined with treatments (e.g. stimulation and/or therapeutic agents) administered using systems and methods disclosed herein. In some embodiments, existing treatments may be administered concurrently with treatments disclosed herein (e.g. stimulation and/or therapeutic agents). In some embodiments, existing treatments may be administered after administration of treatments disclosed herein. In some embodiments, existing treatments may be administered before administration of treatments disclosed herein. In some embodiments, administration of existing treatments to a subject may be separated by a period of time (e.g. at least 1 hour, 5 hours, 8 hours, 24 hours, 2 days, 3 days, 5 days, 1 week, 2 weeks, 1 month, etc.) from administration of treatments disclosed herein. In some embodiments, existing treatments may solely be administered. In some embodiments, existing treatments may be administered using one or more needles of an array of the disclosed systems.

### II. PROVIDED SYSTEMS

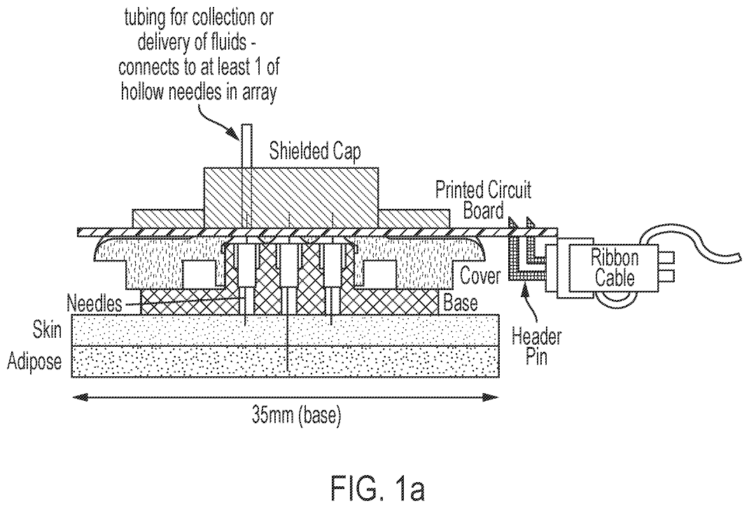

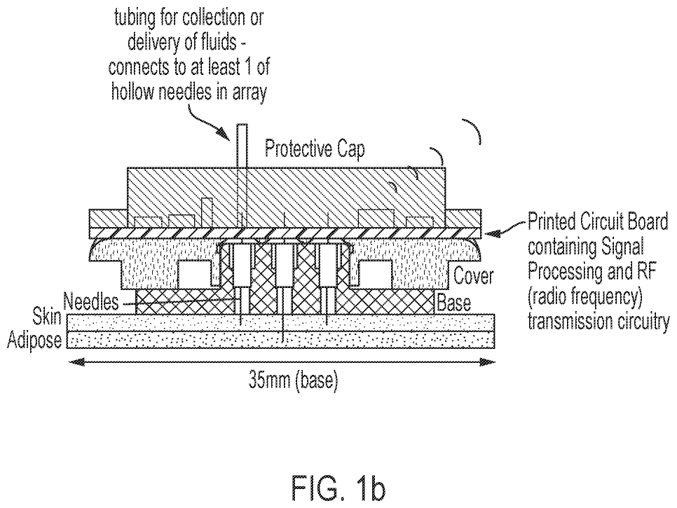

Theragnostic systems (or ‘systems’) of the present disclosure comprise a needle array (for example, a microneedle array), a substrate, and optionally additional components for heating/cooling, stimulation, drug delivery, etc. In some embodiments, systems disclosed herein may be portable. In some embodiments, systems disclosed herein may be benchtop systems. In some embodiments, systems disclosed herein may be wearable systems (e.g. skin adherent material). The footprint or base area of such systems may be more or less than 35 mm×35 mm. In some embodiments, a system/device as described herein may comprise a shielded cap in place to reduce electronic noise and protect the top of the Printed Circuit Board (PCB) to needle connection. In some embodiments, a shielded cap may contain access holes for a needle tubing attachment. In some embodiments, a shielded cap may not contain access holes for a needle tubing attachment. FIG. 1a and FIG. 1b show schematics of the theragnostic device/system as described herein, according to illustrative embodiments. As shown in FIG. 1a, a theragnostic system as described herein, may comprise a needle array (e.g. microneedle array) with wired I/O (input/output). FIG. 1b shows a theragnostic system as described herein that may comprise a needle array (e.g. microneedle array) with wireless I/O (input/output).

In some embodiments, a shielded cap may be made of an electrically conductive material (e.g. a metal, an electrically conductive polymer, etc.). In some embodiments, a shielded cap may be made of an electrically non-conductive material (e.g. a plastic, an electrically non-conductive polymer, etc.). In some embodiments, a shielded cap made of an electrically non-conductive material may be coated with a conductor (e.g. an electrical conductor (e.g. a metal (e.g. copper, aluminum, tin, etc.), a metal alloy (e.g. a copper alloy, etc.))). In some embodiments, a conductor (e.g. electrical conductor) may be a pre-tin shielding. In some embodiments, a shielded cap is grounded (e.g. connected to ground). Such a shielded cap, for example, may be used to shield the electrical connections, components, and/or circuitry from interference (e.g. radio frequency interference).

### Needles and Needle Array:

In accordance with various embodiments, any application-appropriate needle(s) may be used in the systems provided herein. In some embodiments, needles are microneedles (i.e., for example, microneedles may have a length between about 1 μm to about 12,000 μm, or may have a length that may be at most 12,000 μm, or may have a length that may be at least 1 μm). In some embodiments, a needle has a diameter that is consistent throughout the majority of the needle's length. In some embodiments, the diameter of a needle is greatest at the needle's base (i.e., the end opposite, or distal to, the tip). In some embodiments, a needle tapers to a point at the end distal to the needle's base (e.g., to facilitate piercing of a subject's skin). In some embodiments, a needle may be solid. In some embodiments, a needle may be hollow (e.g., through its entire length or a portion thereof). In some embodiments a needle may be tubular. In some embodiments, a needle may be sealed on one or both ends. In some embodiments, a needle is part of an array of needles. In some embodiments, a needle may have a length of between about 1 mm to about 12 mm. In some embodiments, a needle may have a length of between about 1 mm to about 2 mm. In some embodiments, a needle may have a length of between about 2 mm to about 3 mm. In some embodiments, a needle may have a length of between about 3 mm to about 4 mm. In some embodiments, a needle may have a length of between about 4 mm to about 5 mm. In some embodiments, a needle may have a length of between about 5 mm to about 6 mm. In some embodiments, a needle may have a length of between about 6 mm to about 7 mm. In some embodiments, a needle may have a length of between about 7 mm to about 8 mm. In some embodiments, a needle may have a length of between about 8 mm to about 9 mm. In some embodiments, a needle may have a length of between about 9 mm to about 10 mm. In some embodiments, a needle may have a length of between about 10 mm to about 11 mm. In some embodiments, a needle may have a length of between about 11 mm to about 12 mm. In some embodiments, a needle may have a length of greater than about 12 mm. In some embodiments, a needle may have a length of less than about 1 mm. In some embodiments, a needle may have a length of at least 1 mm, at least 2 mm, at least 3 mm, at least 4 mm, at least 5 mm, at least 6 mm, at least 7 mm, at least 8 mm, at least 9 mm, at least 10 mm, at least 11 mm, or at least 12 mm. In some embodiments, a needle may have a length of at most 1 mm, at most 2 mm, at most 3 mm, at most 4 mm, at most 5 mm, at most 6 mm, at most 7 mm, at most 8 mm, at most 9 mm, at most 10 mm, at most 11 mm, or at most 12 mm.

In some embodiments, a needle may be a 32 gauge, 33 gauge, 34 gauge, 35 gauge, 36 gauge, 37 gauge, 38 gauge, 39 gauge, 40 gauge, 41 gauge, or 42 gauge. In some embodiments, a needle may be at least a 32 gauge. In some embodiments, a needle may be at most a 42 gauge. In some embodiments, a needle array may have needles of the same gauge. In some embodiments, a needle array may have needles of different gauges (e.g. one or more gauges, two or more gauges, etc.).

In some embodiments, needles for use in accordance with the present disclosure may be designed and constructed into needle arrays (e.g. microneedle arrays).

### Microneedles (MN) and MN Array:

In some embodiments, microneedles (MN), one or more of which are arranged together to form MN arrays, for use in accordance with the present disclosure are or share features with minimally invasive systems, developed to overcome some of the disadvantages commonly associated with the use of hypodermic and subcutaneous needles, as well as improve patient comfort and compliance. Such disadvantages include, for example, potential for needle tip misplacement with a hypodermic needle because a health professional cannot visualize where exactly the needle is going. Other advantages of MN are that they may not cause bleeding, minimize introduction of pathogens through MN produced holes, and eliminate transdermal dosing variability. Other advantages are the possibility of self-administration, reduce risk of accidental needle stick injuries, reduce risk of transmitting infection, and ease of disposal. In some embodiments, MN are multiple microscopic projections assembled on one side of a support, such as a patch or a device (e.g., stamp, roller, array, applicator, pen).

In some embodiments, microneedles (MN) for use in accordance with the present disclosure may be designed and/or constructed in arrays in order to improve skin contact and facilitate penetration into the skin. In some embodiments, arrays are flexible. In some embodiments, arrays are rigid. In some embodiments, microneedling technologies described herein utilize MN of suitable length, width, and shape to allow for proximity with nerves when inserted into the skin, while still creating efficient pathways for signal detection and drug delivery.

In some embodiments, a suitable MN may be solid, coated, porous, dissolvable, hollow, or hydrogel MN. As disclosed previously, MN arrays or individual MN may be used to delivery treatments for prevention and/or treatment of various diseases, disorders, or conditions, for example neuropathy (e.g. PN). Solid MN create microholes in the skin, thereby increasing transport of a drug formulation (e.g., “poke and patch” methods). Coated MN allow for rapid dissolution of a coated drug into the skin (e.g., “coat and poke” methods). Dissolvable MN allow for rapid and/or controlled release of a drug incorporated within the microneedles. Hollow MN may be used to puncture the skin and enable release of a composition following active infusion or diffusion of a formulation through a microneedle's bores (e.g., “poke and flow” methods”). In the case of dissolvable MN, MN can act as a drug depot, holding a drug composition until released by dissolution in the case of dissolvable MN or swelling in the case of hydrogel MN (e.g., “poke and release” methods).

In some embodiments, a microneedle has a diameter, which is consistent throughout the microneedle's length. In some embodiments, the diameter of a microneedle is greatest at the microneedle's base end. In some embodiments, a microneedle tapers to a point at the end distal to the microneedle's base. In some embodiments, a microneedle may be solid. In some embodiments, a microneedle may be hollow. In some embodiments, a microneedle may be tubular. In some embodiments, a microneedle may be sealed on one end. In some embodiments, a microneedle is part of an array of microneedles. In some embodiments, a microneedle may have an outer diameter of greater than about 100 μm. In some embodiments, a microneedle may have an outer diameter of greater than about 200 μm. In some embodiments, a microneedle may have an outer diameter of less than about 100 μm. In some embodiments, a microneedle may have an outer diameter of less than about 200 μm. In some embodiments, a microneedle may have an outer diameter of about 100 μm, 200 μm, 300 μm, 400 μm, or 500 μm. In some embodiments, a microneedle may have an outer diameter of at most 500 μm. In some embodiments, a microneedle may have an outer diameter of at least 20 μm. In some embodiments, a microneedle may have an inner diameter of greater than about 10 μm. In some embodiments, a microneedle may have an inner diameter of greater than about 20 μm. In some embodiments, a microneedle may have an inner diameter of less than about 10 μm. In some embodiments, a microneedle may have an inner diameter of less than about 20 μm. In some embodiments, a microneedle may have an inner diameter of about 10 μm, 20 μm, 30 μm, 40 μm, or 50 μm. In some embodiments, a microneedle may have an inner diameter of at most 50 μm. In some embodiments, a microneedle may have an inner diameter of at least 1 μm.

In some embodiments, microneedling as described herein comprises applying to skin a plurality of microneedles (e.g., a microneedle array) of common length; in some embodiments, microneedling as described herein comprises applying to skin a plurality of microneedles (e.g., a microneedle array) of different lengths.

Microneedles of various lengths may be used in the microneedling technologies described herein. In some embodiments, the length of the microneedles used as described herein is adjusted based on skin thickness of the treatment site. In some embodiments, a MN or MN array comprises microneedles that may have a length of between about 1 μm to about 12,000 μm. In some embodiments, a MN or MN array comprises microneedles that may have a length of between about 1 μm to about 4,000 μm. In some embodiments, a MN or MN array comprises microneedles that may have a length of between about 1 μm to about 2,000 μm. In some embodiments, a MN or MN array comprises microneedles that may have a length of between about 50 μm to about 400 μm. In some embodiments, a MN or MN array comprises microneedles that may have a length of between about 800 μm to about 1500 μm. In some embodiments, a MN or MN array comprises microneedles of about 50 μm length. In some embodiments, a MN or MN array comprises microneedles of about 100 μm length. In some embodiments, a MN or MN array comprises microneedles of about 150 μm length. In some embodiments, a MN or MN array comprises microneedles of about 200 μm length. In some embodiments, a MN or MN array comprises microneedles of about 250 μm length. In some embodiments, a MN or MN array comprises microneedles of about 300 μm length. In some embodiments, a MN or MN array comprises microneedles of about 350 μm length. In some embodiments, a MN or MN array comprises microneedles of about 400 μm length. In some embodiments, a MN or MN array comprises microneedles of about 450 μm length. In some embodiments, a MN or MN array comprises microneedles of about 500 μm length. In some embodiments, a MN or MN array comprises microneedles of about 550 μm length. In some embodiments, a MN or MN array comprises microneedles of about 600 μm length. In some embodiments, a MN or MN array comprises microneedles of about 650 μm length. In some embodiments, a MN or MN array comprises microneedles of about 700 μm length. In some embodiments, a MN or MN array comprises microneedles of about 750 μm length. In some embodiments, a MN or MN array comprises microneedles of about 800 μm length. In some embodiments, a MN or MN array comprises microneedles of about 850 μm length. In some embodiments, a MN or MN array comprises microneedles of about 900 μm length. In some embodiments, a MN or MN array comprises microneedles of about 950 μm length. In some embodiments, a MN or MN array comprises microneedles of about 1000 μm length. In some embodiments, a MN or MN array comprises microneedles of about 1100 μm length. In some embodiments, a MN or MN array comprises microneedles of about 1200 μm length. In some embodiments, a MN or MN array comprises microneedles of about 1300 μm length. In some embodiments, a MN or MN array comprises microneedles of about 1400 μm length. In some embodiments, a MN or MN array comprises microneedles of about 1500 μm length. In some embodiments, a MN or MN array comprises microneedles of about 2000 μm length. In some embodiments, a MN or MN array comprises microneedles of about 3000 μm length. In some embodiments, a MN or MN array comprises microneedles of about 4000 μm length. In some embodiments, a MN or MN array comprises microneedles of about 5000 μm length. In some embodiments, a MN or MN array comprises microneedles of at least about 50 μm length. In some embodiments, a MN or MN array comprises microneedles of at least about 1 μm, about 10 μm, about 50 μm, about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 2000 μm, about 3000 μm, about 4000 μm, about 5000 μm, about 6000 μm, about 7000 μm, about 8000 μm, about 9000 μm, about 10000 μm, about 11000 μm, about 12000 μm length, about 13000 μm length, about 14000 μm length, about 15000 μm length, about 16000 μm length, about 17000 μm length, about 18000 μm length, about 19000 μm length, about 20000 μm length, about 21000 μm length, about 22000 μm length about 23000 μm length, or about 24000 μm length. In some embodiments, a MN or MN array comprises microneedles of at most about 1 μm, about 10 μm, about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 2000 μm, about 3000 μm, about 4000 μm, about 5000 μm, about 6000 μm, about 7000 μm, about 8000 μm, about 9000 μm, about 10000 μm, about 11000 μm, about 12000 μm length, about 13000 μm length, about 14000 μm length, about 15000 μm length, about 16000 μm length, about 17000 μm length, about 18000 μm length, about 19000 μm length, about 20000 μm length, about 21000 μm length, about 22000 μm length about 23000 μm length, or about 24000 μm length.

In some embodiments, a MN or MN array comprises a plurality of needles. MN arrays for use in accordance with the present disclosure may be fabricated with varying microneedle densities. In some embodiments, a MN or MN array may comprise 2 microneedles/cm². In some embodiments, a MN or MN array may comprise 3 microneedles/cm². In some embodiments, a MN or MN array may comprise 4 microneedles/cm². In some embodiments, a MN or MN array may comprise 5 microneedles/cm². In some embodiments, a MN or MN array may comprise 6 microneedles/cm². In some embodiments, a MN or MN array may comprise 7 microneedles/cm². In some embodiments, a MN or MN array may comprise 8 microneedles/cm². In some embodiments, a MN or MN array may comprise 9 microneedles/cm². In some embodiments, a MN or MN array may comprise 10 microneedles/cm². In some embodiments, a MN or MN array may comprise 11 microneedles/cm². In some embodiments, a MN or MN array may comprise 12 microneedles/cm². In some embodiments, a MN or MN array may comprise 13 microneedles/cm². In some embodiments, a MN or MN array may comprise 14 microneedles/cm². In some embodiments, a MN or MN array may comprise 15 microneedles/cm². In some embodiments, a MN or MN array may comprise 16 microneedles/cm². In some embodiments a MN or MN array, may comprise 17 microneedles/cm². In some embodiments, a MN or MN array may comprise 18 microneedles/cm². In some embodiments, a MN or MN array may comprise 19 microneedles/cm². In some embodiments, a MN or MN array may comprise 20 microneedles/cm². In some embodiments, a MN or MN array may comprise 21 microneedles/cm². In some embodiments, a MN or MN array may comprise 22 microneedles/cm². In some embodiments, a MN or MN array may comprise 23 microneedles/cm². In some embodiments, a MN or MN array may comprise 24 microneedles/cm². In some embodiments, a MN or MN array may comprise 25 microneedles/cm². In some embodiments, a MN or MN array may comprise 26 microneedles/cm². In some embodiments a MN or MN array, may comprise 27 microneedles/cm². In some embodiments, a MN or MN array may comprise 28 microneedles/cm². In some embodiments, a MN or MN array may comprise 29 microneedles/cm². In some embodiments, a MN or MN array may comprise 30 microneedles/cm². In some embodiments, a MN or MN array may comprise 31 microneedles/cm². In some embodiments, a MN or MN array may comprise 35 microneedles/cm². In some embodiments, a MN or MN array may comprise 40 microneedles/cm². In some embodiments, a MN or MN array may comprise 45 microneedles/cm². In some embodiments, a MN or MN array may comprise 50 microneedles/cm². In some embodiments, a MN or MN array may comprise 55 microneedles/cm². In some embodiments a MN or MN array, may comprise 60 microneedles/cm². In some embodiments, a MN or MN array may comprise 65 microneedles/cm². In some embodiments, a MN or MN array may comprise 70 microneedles/cm². In some embodiments, a MN or MN array may comprise 75 microneedles/cm². In some embodiments, a MN or MN array may comprise 80 microneedles/cm². In some embodiments, a MN or MN array may comprise 85 microneedles/cm². In some embodiments, a MN or MN array may comprise 90 microneedles/cm². In some embodiments, a MN or MN array may comprise 95 microneedles/cm². In some embodiments, a MN or MN array may comprise 100 microneedles/cm². In some embodiments, a MN or MN array may comprise 200 microneedles/cm². In some embodiments a MN or MN array, may comprise 300 microneedles/cm². In some embodiments, a MN or MN array may comprise 400 microneedles/cm². In some embodiments, a MN or MN array may comprise 500 microneedles/cm². In some embodiments, a MN or MN array may comprise less than 1000 microneedles/cm². In some embodiments, a MN or MN array may comprise less than 2000 microneedles/cm². In some embodiments, a MN or MN array may comprise at most about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 35, about 40, about 45, about 50, about 55, about 60, about 65, about 70, about 75, about 80, about 85, about 90, about 95, about 100, about 200, about 300, about 400, about 500, about 1000, or about 2000 microneedles/cm². In some embodiments, a MN or MN array may comprise at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 35, about 40, about 45, about 50, about 55, about 60, about 65, about 70, about 75, about 80, about 85, about 90, about 95, about 100, about 200, about 300, about 400, about 500, about 1000, or about 2000 microneedles/cm².

Microneedles of any shape may be used in the microneedling technologies described herein. In some embodiments, microneedles may have a circular cross-section. In some embodiments, microneedles may have a triangular cross-section. In some embodiments, microneedles may have a rectangular cross-section. In some embodiments, microneedles may have a square cross-section. In some embodiments, microneedles may have a quadrangular cross-section. In some embodiments, microneedles may have a pentagular cross-section. In some embodiments, microneedles may have a hexangular cross-section. In some embodiments, microneedles may have a septangular cross-section. In some embodiments, microneedles may have an octangular cross-section. In some embodiments, microneedles may have a nonangular cross-section. In some embodiments, microneedles may have a decangular cross-section.

In some embodiments, a MN in accordance with the present disclosure may be coated (e.g., partially or completely). As a person of ordinary skill in the art may be aware, coatings may aid needles in penetrating into epidermis, dermis, hypodermis, or further beneath the skin such as to adipose or muscle tissue. In some embodiments, a MN may be coated with a single material. In some embodiments, a MN may be coated with two or more materials. In some embodiments, a MN may be coated with a first coating. In some embodiments, a MN may be coated with two or more coatings. In some embodiments, a MN may be coated on the outside. In some embodiments, a MN may be coated on the inside. In some embodiments, a MN may be coated on the inside and outside. In some embodiments, a MN may be coated with a lubricant. In some embodiments, a MN may be coated with an electrically conductive material. In some embodiments, a MN may be coated with stainless steel, silicon, platinum, gold, silver, copper, or any combination thereof. In some embodiments, one or more coatings may be to insulate a signal. In some embodiments, a MN may be coated with stainless steel. In some embodiments, a MN may be coated with an electrical insulator.

In some embodiments, a MN in accordance with the present disclose may be filled with one or more materials. In some embodiments, one or more MN may be at least partially filled. In some embodiments, one or more MN may be completely filled. In some embodiments, a MN may be at least partially filled. In some embodiments, a MN may be completely filled. In some embodiments, a MN may be filled with a filling material. In some embodiments, filling material is an electrically conductive material. In some embodiments, an electrically conductive material may be a metal. In some embodiments, an electrically conductive material is stainless steel, silicon, platinum, gold, silver, copper, or any combination thereof. In some embodiments, an electrically insulating material may be a polymer. In some embodiments, a filling material may be an electrically insulating material. In some embodiments, filling material may be in the form of a liquid, a gel, an emulsion, a lotion, a cream, a fluid, or a solid.

In some embodiments, a MN for use in accordance with the present disclosure may be fabricated from different materials. In some embodiments, a MN may be manufactured using various types of biocompatible materials including polymers, metal, ceramics, semiconductors, organics, composites, or silicon. In some embodiments, a MN may be manufactured using a metal. In some embodiments, a MN is manufactured using silicon, platinum, gold, silver, copper, any electrically conductive metal, or any combination thereof. In some embodiments, a MN is manufactured using silicon. In some embodiments, a MN is manufactured using stainless steel. Unless they are designed to break off into the skin and dissolve, in some embodiments, microneedles have the mechanical strength to remain intact and to record nerve conductance, deliver drugs, or collect biological fluid, while being inserted into the skin and/or removed from the skin after insertion. In some embodiments MN are capable of remaining in place for up to a number of days before intact removal. In some embodiments, microneedles may be sterilizable using standard technologies. In some embodiments, a MN may be biodegradable. In some embodiments, a MN may comprise a polymeric material. In some embodiments the polymeric material comprises poly-L-lactic acid, poly-glycolic acid, poly-carbonate, poly-lactic-co-glycolic acid (PLGA), polydimethylsiloxane, polyvinylpyrrolidone (PVP), a copolymer of methyl vinyl ether and maleic anhydride, sodium hyaluronate, carboxymethyl cellulose, maltose, dextrin, galactose, starch, gelatin, polypyrrole, polyacetylene, polyaniline, or a combination thereof. In some embodiments, MN are disposable. In some embodiments, MN are reusable.

In some embodiments, MN for use in accordance with the present disclosure may be fabricated using technologies including, but not limited to 3D-printing, micro-molding processes or lasers. In some embodiments, MN may be fabricated using 3D-printing.

In some embodiments, MN for use in accordance with the present disclosure may have different functions. In some embodiments, a MN may be used as an electrode. In some embodiments, a MN may be used as a reference electrode. In some embodiments, a MN may be used as a ground electrode. In some embodiments, a MN may be used as a stimulating electrode. In some embodiments, a MN may be used to deliver treatments (e.g. therapeutic agents and/or stimulation) to treat a disease, disorder, or condition (for example, neuropathy (e.g. PN)). In some embodiments, a MN may be used for sampling biological samples. In some embodiments, a MN may be used for sampling biological fluids. In some embodiments, a MN may be used for fluid transport. In some embodiments, fluid transport may be bidirectional. In some embodiments, fluid transport may be unidirectional. In some embodiments, fluid transport may be from disclosed systems (e.g. needles) to skin or other tissue. In some embodiments, fluid transport may be from skin or other tissue to disclosed systems (e.g. needles). In some embodiments, fluid transport is between disclosed systems (e.g. needles) and one or more of skin, interstitial fluid, blood, sweat, and/or subcutaneous tissue of a subject. In some embodiments, a MN may be used to deliver treatments. In some embodiments, delivery is achieved transdermally. In some embodiments, delivery is achieved sub-dermally. In some embodiments, delivery is achieved subcutaneously.

### Substrates:

Needle arrays and/or MN arrays for use in accordance with the present disclosure may be fabricated on a substrate. In some embodiments, a substrate may have different surface areas. In some embodiments, a substrate may have a surface area of at least about 1 square inch. In some embodiments, a substrate may have a surface area of at least about 2 square inches. In some embodiments, a substrate may have a surface area of at least about 3 square inches. In some embodiments, a substrate may have a surface area of at least about 4 square inches. In some embodiments, a substrate may have a surface area of at least about 5 square inches. In some embodiments, a substrate may have a surface area of at least about 10 square inches. In some embodiments, a substrate may have a surface area of less than 1 square inch.

In some embodiments, a substrate may have at least two dimensions. In some embodiments, at least one dimension may be at least about 1 inch. In some embodiments, at least one dimension may be at least about 2 inches. In some embodiments, at least one dimension may be at least about 2 inches. In some embodiments, at least one dimension may be at least about 4 inches. In some embodiments, at least one dimension may be at least about 5 inches. In some embodiments, at least one dimension may be at least about 6 inches. In some embodiments, at least one dimension may be at least about 7 inches. In some embodiments, at least one dimension may be at most about 8 inches.

In some embodiments, needle arrays and/or MN arrays for use in accordance with the present disclosure may be fabricated one a substrate with different shapes. Substrate shapes may be fabricated in order to accommodate various body parts or sizes of different subjects and/or patients. In some embodiments, a substrate may be shaped as a circle or a polygon. In some embodiments, a substrate may be shaped as a circle, triangle, square, rectangle, pentagon, quadrilateral, hexagon, octagon, or any other shape. In some embodiments, a substrate may be shaped as a square. In some embodiments, a substrate may be shaped as a rectangle.

Substrates for use in accordance with the present disclosure may be fabricated from different materials. In some embodiments, a substrate may be fabricated using the same material as a MN array or needle array. In some embodiments, a substrate may be fabricated using a different material from a MN array or needle array. In some embodiments, a substrate is flexible. In some embodiments, a substrate is rigid. In some embodiments, a substrate may be manufactured using various types of biocompatible materials including polymers, metal, ceramics, semiconductors, organics, composites, or silicon. In some embodiments, a substrate may be capable of remaining in place for up to a number of days before intact removal. In some embodiments, a substrate may be biodegradable. In some embodiments, a substrate may comprise a polymeric material. In some embodiments, the polymeric material comprises poly-L-lactic acid, poly-glycolic acid, poly-carbonate, poly-lactic-co-glycolic acid (PLGA), polydimethylsiloxane, polyvinylpyrrolidone (PVP), a copolymer of methyl vinyl ether and maleic anhydride, sodium hyaluronate, carboxymethyl cellulose, maltose, dextrin, galactose, starch, gelatin, polypyrrole, polyacetylene, polyaniline, or a combination thereof.

Substrates for use in accordance with the present disclosure may be fabricated using technologies including, but not limited to 3D-printing, micro-molding processes or lasers. In some embodiments, substrates are fabricated using 3D-printing.

### Additional Components:

Systems as described herein may comprise additional components to augment their diagnostic and therapeutic functionalities. In some embodiments, additional components are optional.

In some embodiments, systems as described herein comprise a means for providing fluid flow, which is fluidically coupled to at least one needle (e.g. microneedle) of disclosed systems. For example, in some embodiments, systems as described herein may comprise one or more reservoirs to store one or more therapeutic agents (e.g. drug therapies, biological agents, etc.), which is fluidically connected to needles of the device for sustained and timely release of one or more therapeutic agents as and when needed. In some embodiments, a means for providing fluid flow is a syringe pump. In some embodiments, a means for providing fluid flow is a mechanical pump.

In some embodiments, substrates are configured to allow adjustment of the depth of penetration of one or more needles into skin and/or subcutaneous tissue of a subject. In some embodiments, systems disclosed herein may comprise a spacer. In some embodiments, a space is adjustable. That is, in some embodiments, a spacer may aide in controlling the depth of penetration of one or more needles (e.g. microneedles). Accordingly, the dimensions of a spacer depend on the requisite depth of penetration of needles. In some embodiments, a spacer has a thickness of at least 50 μm. In some embodiments, a spacer is at least about 500 μm thick. In some embodiments, a spacer is at most about 5000 μm thick. In some embodiments, a spacer is at most about 12000 μm thick. A person of ordinary skill in the art would appreciate that a spacer may have a thickness appropriate to adjust the depth of penetration of needles. Accordingly, in some embodiments, a spacer could have a thickness that varies or is in the range from zero to 12 mm. In some embodiments, a spacer may have a thickness of about 50 μm to about 2 mm.

In some embodiments, systems disclosed herein may comprise a cooling mechanism. For example, cooling may be achieved through chemical or electronic means. In some embodiments, cooling is achieved using methanol. In some embodiments, cooling is achieved using a topical formulation. In some embodiments, a formulation is a cream or lotion. In some embodiments, cooling is achieved using a fan. In some embodiments, cooling is achieved using electrical, thermoelectric, or pyroelectric means. In some embodiments, cooling is achieved using cooling coil (e.g., with water flowing through small tube).

In some embodiments, systems disclosed herein may comprise a mechanism of stimulation of nerves to revive functionality in such nerves or stimulate nerve regrowth. In some embodiments, stimulation may be achieved using one or more needles of systems disclosed herein. In some embodiments, stimulation is mechanical. In some embodiments, stimulation is achieved by vibration (e.g. vibration of needles) and systems disclosed herein may comprise a device component that enables generation of vibrational motion in one or more needles. In some embodiments, the device component may be a motor. In some embodiments, stimulation is achieved by temperature regulation (e.g. temperature regulation of needles) and systems may comprise a device component that enables temperature regulation. In some embodiments, the device component may be an additional flexible backing. In some embodiments, a flexible backing may be used to provide cold stimulation. In some embodiments, the device component may be a cooling coil.

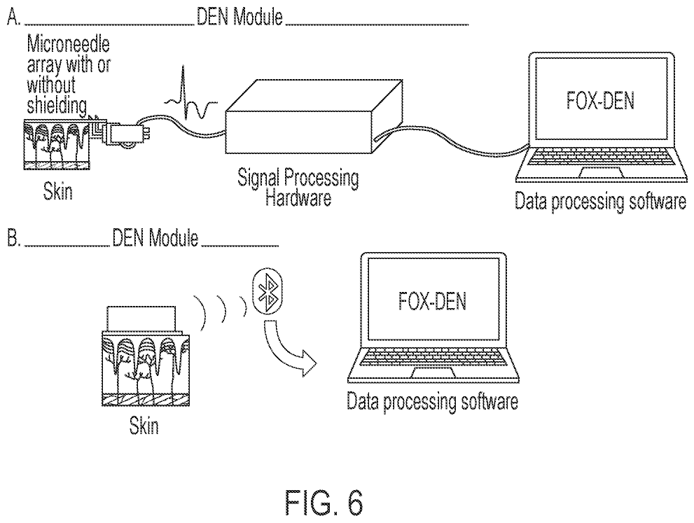

Systems as described herein may also comprise electronics for recording signals, for example from nerve fibers. In some embodiments, systems may comprise one or more digital processors and associated electronics configured to receive data from a device (e.g., needles) and/or system components. In some embodiments, systems may comprise one or more digital processors and associated electronics configured to transmit data to a device (e.g., needles) and/or system components. This will allow for systems as disclosed herein to be employed in various clinical settings, eliminating the need for patients to be screened by a specialist. In some embodiments, associated software (e.g. FOX-DEN) may be provided to integrate nerve recording data with interpretation of neuropathic state in a simple FDA/HIPPA-compliant interface, decreasing the need for specialized training in understanding nerve recording data. FIG. 6 shows a schematic of a system for use in on-site processing including signal amplification and wireless communication between system/device disclosed herein (termed ‘DEN Module’ in FIG. 6) and a computing device.

In some embodiments, systems may comprise a power source to provide power. In some embodiments, a power source may be a battery. In some embodiments, systems may comprise a PC-card and/or an amplifier. In some embodiments, systems may comprise a wireless transmission module to wirelessly exchange data to and from an operating device. This allows for the systems described herein to be portable and employed in various clinical settings, eliminating the need for patients to be screened by a specialist.

In some embodiments, systems may comprise one or more sensors to analyze biological samples. In some embodiments, one or more sensors may be electrochemical sensors. In some embodiments, one or more sensors may be electronic (e.g. Field Effect Transistor (FET), Ion channel, etc.).

### Manufacturing:

The technologies disclosed in the present application may be manufactured using any known manufacturing techniques in the art. In some embodiments, systems as disclosed herein may be manufactured using microfabrication technology. In some embodiments, systems as disclosed herein may be manufactured using 3D printing.

In some embodiments, all components of systems described herein may be manufactured using a single manufacturing process. In some embodiments, a subset of components of systems described herein may be manufactured using a single manufacturing process. In some embodiments, components of systems may be manufactured using micfabrication technology. In some embodiments, components of systems are manufactured using 3D-printing.

In some embodiments, needles (e.g. microneedles) of systems disclosed herein may be manufactured using micfabrication technology. In some embodiments, needles may be manufactured by chemical isotropic etching. In some embodiments, needles may be manufactured by injection moulding. In some embodiments, needles may be manufactured by reactive ion etching. In some embodiments, needles may be manufactured by surface micromachining. In some embodiments, needles may be manufactured by bulk micromachining. In some embodiments, needles may be manufactured by micromolding. In some embodiments, needles may be manufactured by lithography-electroforming-replication. In some embodiments, needles may be manufactured by laser drilling. See Trichur et al., 2002; Yang & Zahn, 2004; Davis et al., 2005; Moon et al., 2005; Park et al., 2005; Stoeber & Liepmann, 2005, which are incorporated herein by reference. In some embodiments, needles may be manufactured by electroplating. In some embodiments, needles may be manufactured by photochemical etching. In some embodiments, needles may be manufactured by laser cutting. See Chandrasekaran & Frazier, 2003; Chandrasekaran et al., 2003; Verbaan et al., 2008, which are incorporated herein by reference. In some embodiments, needles of systems disclosed herein may be manufactured using 3D-printing.

As discussed in the previous sections various materials may be used to construct components of systems disclosed herein. In some embodiments, one or more materials may be used to construct systems components disclosed herein. In some embodiments, one material may be used to construct systems components disclosed herein. In some embodiments, one or more materials used to construct system components may be biocompatible. In some embodiments, one or more materials used to construct system components may be biodegradable. For example, needles (e.g. microneedles) of the present disclosure may be constructed using various materials. In some embodiments, needles may be constructed using various types of one or more biocompatible materials including polymers, metal, ceramics, semiconductors, organics, composites, or silicon. In some embodiments, needles may be constructed using a semiconductor. In some embodiments, needles may be constructed of silicon. In some embodiments, needles may be constructed using metals. In some embodiments, needles may be constructed using one or more of stainless-steel, titanium, palladium, palladium-cobalt alloys, nickel, platinum, gold, silver, copper, any electrically conductive metal, or any combination thereof. In some embodiments, needles are constructed using stainless steel. In some embodiments, needles may comprise a polymeric material. In some embodiments, polymeric material comprises poly-L-lactic acid, poly-glycolic acid, poly-carbonate, poly-lactic-co-glycolic acid (PLGA), polydimethylsiloxane, polyvinylpyrrolidone (PVP), a copolymer of methyl vinyl ether and maleic anhydride, sodium hyaluronate, carboxymethyl cellulose, maltose, dextrin, galactose, starch, gelatin, polypyrrole, polyacetylene, polyaniline, or a combination thereof. In some embodiments, needles may be microneedles.

Recording electronics of systems as described herein may be manufactured using fabrication technologies known in the art. In some embodiments, recording electronics may be fabricated using traditional microfabrication technologies. In some embodiments, recording electronics may be fabricated by chemical isotropic etching. In some embodiments, recording electronics may be fabricated by injection moulding. In some embodiments, recording electronics may be fabricated by reactive ion etching. In some embodiments, recording electronics may be fabricated by surface micromachining. In some embodiments, recording electronics may be fabricated by bulk micromachining. In some embodiments, recording electronics may be fabricated by micromolding. In some embodiments, recording electronics may be fabricated by lithography-electroforming-replication. In some embodiments, recording electronics may be fabricated by laser drilling. In some embodiments, recording electronics may be fabricated and/or assembled on a printed circuit board (PCB). As is known to a person of ordinary skill in the art, electronic components used in a PCB may be off-shelf components.

### III. SKIN

Aspects of present disclosure relates to interaction of disclosed systems with skin and/or various components of skin. As is known to a person of ordinary skill in the art, human skin is largest organ of the body and helps protect from microbes and pathogens, regulates body temperature, protects from excessive water loss, synthesizes vitamin D, and permits sensations such as touch, heat, cold, and pain.

### Components:

Human skin comprises multiple layers, including the epidermis, and dermis. The epidermis has several layers of tissue, namely, stratum corneum, stratum lucidum, stratum granulosum, stratum spinosum, and stratum basale (identified in order from the outer surface of the skin inward).

The stratum corneum presents the most significant hurdle in transdermal delivery of medications. The stratum corneum is typically about 10 μm-15 μm thick, and it comprises flattened, keratised cells (corneocytes) arranged in several layers. The intercellular space between the corneocytes is filled with lipidic structures, and may play a role in the permeation of substances through skin (Bauerova et al., 2001, Eur. J. Drug Metabolism Pharmacokinetics, 26:85; incorporated herein by reference). The rest of the epidermis below the stratum corneum is approximately 150 μm thick.

The dermis is about 1 mm-2 mm thick and is located below the epidermis. The dermis is supported by various capillaries as well as neuronal processes and consists of connective tissue and cushions the body from stress and strain. The dermis is structurally divided into two areas: a superficial area adjacent to the epidermis, called the papillary region, and a deep thicker area known as the reticular region.

The hypodermis or the subcutaneous tissue is not part of the skin but lies below the dermis. Its purpose is to attach the skin to underlying bone and muscle as well as supplying it with blood vessels and nerves. It consists of connective tissue, adipose tissue, and elastin, and comprises fibroblasts, macrophages, and adipocytes.

Interaction with Provided Systems:

As disclosed previously, systems disclosed herein can interact with one or more layers of the skin and/or the subcutaneous tissue. For example, needles of systems disclosed herein may be fabricated with specific lengths so that they interact with specific layers of the skin and/or subcutaneous tissue. In some embodiments, a spacer may be used to adjust the depth of penetration of needles of systems disclosed herein.

In some embodiments, detection (e.g. monitoring, recording etc.) of signals may be performed in any one of the layers of the skin. In some embodiments, detection may be performed in epidermis. In some embodiments, detection may be performed in dermis. In some embodiments, detection may be performed in subcutaneous tissue.

In some embodiments, sampling of biological samples may be obtained from any one of the layers of the skin. In some embodiments, samples may be obtained from epidermis. In some embodiments, samples may be obtained from dermis. In some embodiments, samples may be obtained from subcutaneous tissue.

In some embodiments, treatment (e.g. therapeutic agents, stimulation, etc.) may be administered to any one of the layers of the skin. In some embodiments, treatment may be administered to epidermis. In some embodiments, treatment may be administered to dermis. In some embodiments, treatment may be administered to subcutaneous tissue.

In some embodiments, needles of varying length may be used to interact (e.g., detect, sample, stimulate, administer treatment etc.) with more than one layer of skin and/or subcutaneous tissue. In some embodiments, such interaction with one or more layers is simultaneous. In some embodiments, such interaction is serial (i.e. one interaction after another).

### IV. DISEASES, DISORDERS, AND CONDITIONS

### Peripheral Neuropathy:

Peripheral neuropathy (PN) is a devastating condition affecting patients, their families, and society as a whole. PN refers to the conditions that result when nerves that carry signals to and from the brain and spinal cord from and to the rest of the body are damaged or diseased. The peripheral nerves are an intricate network of nerves that connect the brain and spinal cord to muscles and organs. These nerves come out of the spinal cord and are arranged along lines in the body called dermatomes. Damage to a nerve affects one or more dermatomes and can be tracked to specific areas in the body. Typically, nerve damage leads to interrupted communication between the brain and other parts of the body. It can also impair muscle movement, prevent normal sensation in arms and legs (for example, cause burning sensation in the feet or hands), cause tingling, numbness, and/or pain.

According to a recent FDA report, The Voice of the Customer, patients sometimes experience PN unpredictably or suddenly and can struggle daily with their PN symptoms, thus limiting their ability to seek adequate and consistent pain relief. Many patients describe its impact as loss or significant changes to their careers, limited social interactions, decreased quality time with family, and feelings of hopelessness due to their disease (see FDA, U.S.F.a.D.A., Neuropathic Pain Associated with Peripheral Neuropathy, FDA, Editor. 2017).

An estimated 30 million people in the US alone are affected by some form of PN according to The Foundation for Peripheral Neuropathy, including: diabetic peripheral neuropathy (DPN), chemotherapy-induced peripheral neuropathy (CIPN); HIV or AIDS-induced neuropathy; and idiopathic peripheral neuropathies (IPN), neuropathies with no identifiable known cause (such as with aging). Currently, patients are only treated with analgesics or pain medications, which cannot mitigate the range of symptoms that include numbness, tingling, hypersensitivity, and weakness, or in more severe cases limb amputation (due to tissue necrosis). Although there are situations in which intervention can slow the progression of PN, there is no therapy on the market to completely halt and reverse the neurodegeneration.

Diabetic Peripheral Neuropathy (DPN): DPN is known to occur in patients with diabetes. DPN represents the largest category of PN and involves ‘dying-back’ of axons in the skin of distal extremities, leading to loss of nerve conduction as well as pain and discomfort. Symptoms include numbness, tingling, tactile hypersensitivity, loss of thermal perception, weakness, impaired coordination, and loss of reflexes. The International Diabetes Federation estimates that 463 million individuals have diabetes, and this disease accounts for 10% ($760 billion USD) of global health expenditures (see Federation, I.D., IDF Diabetes Atlas—9th edition. 2019, International Diabetes Federation: Online). It is estimated that over 60-70% of diabetes patients have DPN. However, these numbers are likely significantly underestimated; recent research has demonstrated that pre-diabetic patients show signs of PN, and it is also suggested that there is also a ‘healthy’ population with early signs of neuropathy but with no dependable tool to diagnose it so early. Diabetes and other metabolic diseases also increase in incidence with age and with obesity and given that lifespan has increased for the latest aging population, it is expected that incidences of PN will continue to rise.

Chemotherapy-Induced Peripheral Neuropathy (CIPN): Following diabetes, the next leading cause of PN is CIPN with 30-40% of all cancer patients presenting with the condition. Chemotherapy is detrimental to the nervous system because nerve cells are more sensitive than other cells in the body. Sensory nerves (which are responsible for sensation, including pain) are more highly affected and at risk than motor nerves. With CIPN, symptoms may occur immediately after a dose of chemotherapy or have a delayed onset up to weeks, months or years after treatment is completed (see Neuropathy, T.F.f.P., Peripheral Neuropathy Risk Factors+Facts. 2019, The Foundation for Peripheral Neuropathy: Online).

AIDS-Induced Neuropathy: HIV or AIDs patients also often develop PN and the CDC estimates that 20-50% of these individuals have neuropathy.

Idiopathic Peripheral Neuropathies (IPN): Idiopathic peripheral neuropathies account for the third leading cause of PN, representing 23% of all neuropathy patients. IPN is often seen in middle-aged and older people. According to Martyn et al. 1997 (see Martyn, C. N. and R. A. Hughes, Epidemiology of peripheral neuropathy. J Neurol Neurosurg Psychiatry, 1997. 62(4): p. 310-8), the prevalence of PN rises by nearly 6% in people older than 55 years of age. Altogether, there are over 30 conditions that can lead to neuropathy. These figures do not include traumatic peripheral nerve injuries, and as England and Asbury, 2004 (see England, J. D. and A. K. Asbury, Peripheral neuropathy. Lancet, 2004. 363(9427): p. 2151-61) stated, “the total burden of peripheral neuropathy on society is even greater”.

Auto-Immune Neuropathies: Patients of Autoimmune disease, i.e. a disease in which the immune system mistakenly attacks the patient's own body, may also suffer from damage to their nerves. In auto-immune neuropathies, the immune system directly targets nerves or the surrounding tissues that compress or entrap nerves. Examples of such diseases include Sjogren's syndrome, systemic lupus erythematosus, rheumatoid arthritis and celiac disease. Guillain-Barre syndrome is an autoimmune disease that happens rapidly and can affect autonomic nerves.

Other Virus-related Neuropathies: Patients suffering from other viral diseases and/or condition (e.g. COVID-19) may also develop PN. For example, viruses such as varicella-zoster virus (which causes chicken pox and shingles), West Nile virus, cytomegalovirus, and herpes simplex target sensory fibers, causing attacks of sharp, lightning-like pain.

### Diagnosis:

PN is currently diagnosed by a variety of medical practitioners. However, only PN specialists are capable of providing diagnosis based on nerve function analysis. Besides physical exams that include blood tests, neuropathies like PN is typically diagnosed by neurological examinations and by performing one or more of imaging tests, nerve function tests, nerve biopsies, and skin biopsies. Blood tests can detect vitamin deficiencies, abnormal immune function and other indications of conditions that can cause PN. Imaging tests (e.g. CT or MRI scans) can aide in visualize herniated disks, tumors or other abnormalities that may cause nerve damage leading to neuropathy. Nerve function tests like an autonomic reflex screen that records how the autonomic nerve fibers work, or a sweat test that measures a body's ability to sweat enable a physician to record and diagnose nerve functionality. A common nerve function tests is the Electromyography (EMG) that records electrical activity of muscles to detect nerve damage. In an EMG a thin needle (electrode) is inserted into the muscle to measure electrical activity as the muscle is contracted. At the same time as an EMG, typically nerve conduction studies are also performed in which flat electrodes are placed on the skin and a low electric current stimulates the nerves. Responses to the stimulation are then recorded and used to diagnose nerve damage. Finally, nerve and skin biopsies may be performed to look for abnormalities in a nerve and to look for reduction in nerve endings in the skin. The common disadvantages of each of these tests and methods are that they are laborious, time-consuming, expensive, and most importantly invasive and painful, causing extreme discomfort to the patient. Furthermore, these tests typically are able to measure only large changes in nerve functionality due to which detection of neuropathies occur at very late stages leading to poor disease management.

Table 1 provides a summary of PN clinicians' opinions from dozens of clinician interactions regarding current limitations in standard of care with respect to PN diagnosis and treatment. This reiterates the need for a reliable early diagnostic tool for PN that is rooted in nerve function assessment. As confirmed by Table 1, most diagnoses of neuropathy occur at specialized clinics, and tests are almost exclusively administered by neurologists. As listed in Table 1, an ideal system would enable diagnosis to be performed as part of a routine physical by general practitioners and nurses. Having a neuropathy diagnosis tool that can be used by non-neurological specialists would remove some burden from neurologists and reduce the time-to-diagnosis for the patient, allowing for faster implementation of treatment interventions.

TABLE 1
Customer Discovery Keypoints

| | |
|---|---|
| Limitations in Standard | lack of reliable early diagnostic tools |
| of Care | diagnostic tools are aimed a larger nerves, which die back late in a disease state |
| (early and accurate | qualitative assessments problematic (false negatives, false positives) |
| diagnosis of peripheral | inability to assess structure AND function |
| neuropathy) | current methods not very specific to nerve subtypes and not very reproducible |
| | no ability to measure adipose tissue neuropathy |
| Limitations In Standard | all treatments aimed at pain relief or reversal of causative conditions (diabetes |
| of Care | mitigation, discontinue antibiotic or chemotherapy) |
| (treatment of peripheral | intervention strategy (i.e. controlling blood sugar in diabetics) can only be |
| neuropathy) | implemented if diagnosis is early |
| Ideal Diagnostic | simple and quick, can be performed in clinic |
| | easy to train personnel to use |
| | direct assessment that eliminates qualitative input |
| | inexpensive ($100-200) |
| | early & accurate diagnostic would change current standard of care |

As disclosed above, current methods for diagnosing PN in the clinic are either based on subjective patient reporting of sensations or rely on indirect measures of neuropathic pain or loss of sensory function (i.e., sweat gland, vibration, etc.). Diagnostics that rely on nerve conductance measurements provide a clear functional assessment of nerve fibers but are invasive, painful, and technically advanced techniques are required to measure large nerve function. There are currently no products on the market that allow for sensitive measurements of nerve conduction in small nerve fibers. In addition, there are no products on the market that allow for the other capabilities, including drug delivery and other forms of nerve growth stimulation.

### Treatment:

While no treatment for neuropathy exits, treatment goals are to manage the condition causing neuropathy and to relieve symptoms. Typical medications prescribed to neuropathy patients include: pain relievers, including medications containing opioids, such as tramadol (Conzip, Ultram) or oxycodone (Oxycontin, Roxicodone, others), which can lead to dependence and addiction; topical treatments such as capsaicin creams, lidocaine patches etc. that may cause irritation, skin burning, dizziness or drowsiness in patients; antidepressants that have been found to relieve pain but that can lead to addiction and have various other side effects. While side effects for each of these medications are well known, none of these can treat neuropathy and only help alleviate its symptoms. To this end various therapies and procedures might help to further ease the symptoms of neuropathy. These include Transcutaneous electrical nerve stimulation (TENS), plasma exchange and intravenous immune globulin, physical therapy, and/or surgery. However, each of these therapies are expensive, time consuming, and/or invasive leading to decreased quality of life in patients and no permanent treatment to patients' condition.

As of 2014, it was estimated that costs of neuropathy pain management drugs were over $2000 per patient (see Schaefer, C., et al., Pain severity and the economic burden of neuropathic pain in the United States: BEAT Neuropathic Pain Observational Study. Clinicoecon Outcomes Res, 2014. 6: p. 483-96); this represents a doubling since 2007 (see Barrett, A. M., et al., Epidemiology, public health burden, and treatment of diabetic peripheral neuropathic pain: a review. Pain Med, 2007. 8 Suppl 2: p. S50-62). The devices, systems, and methods as disclosed herein, will provide relief in the form of indirect non-medical costs estimated at over $19,000 per patient per year (see Schaefer, C., et al., Pain severity and the economic burden of neuropathic pain in the United States: BEAT Neuropathic Pain Observational Study. Clinicoeconomic Outcomes Res, 2014. 6: p. 483-96); this includes loss of productivity in individuals who are afflicted by this condition. Most importantly, none of the current devices on the market (or under research) are designed to diagnose and treat PN. Not only does the technology presented herein provide early detection, it is designed to deliver drugs for treatment, and collect biological samples (i.e. interstitial fluid) which could lead to the identification of one or more biomarkers for this condition, further bolstering the nerve conductance diagnostic aspect of the disclosed system. A new biomarker would allow a better understanding of implicated pathways and the development of new therapeutics, as well as the potential for an interstitial diagnosis alone using a test for the biomarker. The technologies as disclosed herein would thus be a first-to-market theragnostic for early detection of PN. Furthermore, once therapeutic agents are developed the technologies presented herein may then be used to treat PN at the point when disease progression may be slowed and possibly even reversed.

Technologies provided herein may be used for diagnosing, evaluating, treating and/or preventing any of a variety of systemic diseases, disorders, and/or conditions. In some embodiments, the present disclosure provides technologies for diagnosing, treating and/or preventing diseases, disorders, or conditions which are systemic. In some embodiments, the present disclosure provides technologies for diagnosing, treating and/or preventing diseases, disorders or conditions associated with the epidermal and/or dermal level of the skin. In some embodiments, the present disclosure provides technologies for diagnosing, treating and/or preventing diseases, disorders, or conditions associated with degradation of the peripheral nerves. In some embodiments, the present disclosure provides technologies for diagnosing, treating and/or preventing diseases, disorders, or conditions associated with neuropathy. In some embodiments, the present disclosure provides technologies for diagnosing, treating and/or preventing diseases, disorders, or conditions associated with peripheral neuropathy. In some embodiments, the present disclosure provides technologies for diagnosing, treating and/or preventing diseases, disorders, or conditions associated with diabetic neuropathy. In some embodiments, the present disclosure provides technologies for diagnosing, treating and/or preventing diseases, disorders, or conditions associated with diabetic peripheral neuropathy (DPN). In some embodiments, the present disclosure provides technologies for diagnosing, treating and/or preventing diseases, disorders, or conditions associated with cancer. In some embodiments, the present disclosure provides technologies for diagnosing, treating and/or preventing diseases, disorders, or conditions associated with chemotherapy-induced neuropathy (CIPN). In some embodiments, the present disclosure provides technologies for diagnosing, treating and/or preventing diseases, disorders, or conditions associated with HIV. In some embodiments, the present disclosure provides technologies for diagnosing, treating and/or preventing diseases, disorders, or conditions associated with AIDS-induced neuropathy. In some embodiments, the present disclosure provides technologies for diagnosing, treating and/or preventing diseases, disorders, or conditions associated with idiopathic peripheral neuropathies (IPN).

### Administration of Treatment:

In some embodiments, technologies disclosed herein include administration of at least one treatment, for example therapeutic agents and/or stimulation, administered using systems as described herein, according to a dosing regimen sufficient to achieve a reduction in the degree and/or prevalence of a relevant neuropathic condition of at least about 20%; in some embodiments according to a dosing regimen sufficient to achieve a of at least about 25%; in some embodiments according to a dosing regimen sufficient to achieve a reduction of at least about 30%; in some embodiments according to a dosing regimen sufficient to achieve a reduction of at least about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, or more.

In some embodiments, technologies disclosed herein involves administration of at least one treatment, administered using the system as described herein, according to a dosing regimen sufficient to achieve a reduction in the degree and/or prevalence of a relevant neuropathic condition of at least about 20% in a specified percentage of a population of patients to which the treatment was administered; in some embodiments according to a dosing regimen sufficient to achieve a of at least about 25% in a specified percentage of a population of patients to which the treatment was administered; in some embodiments according to a dosing regimen sufficient to achieve a reduction of at least about 30% in a specified percentage of a population of patients to which the treatment was administered; in some embodiments according to a dosing regimen sufficient to achieve a reduction of at least about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90% or more in a specified percentage of a population of patients to which the treatment was administered. In some embodiments, the specified percentage of population of patients to which the treatment was administered is at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. To give but a few illustrative examples, in some embodiments, technologies disclosed herein involves administration of at least one provided treatment according to a dosing regimen sufficient to achieve a reduction in the degree and/or prevalence of a relevant neuropathic condition of at least about 20% in at least about 50% of the population of patients to which the treatment was administered. In some embodiments, technologies disclosed herein involves administration of at least one provided treatment according to a dosing regimen sufficient to achieve a reduction in the degree and/or prevalence of a relevant neuropathic condition of at least about 30% in at least about 50% of the population of patients to which the treatment was administered.

In some embodiments, provided treatments comprise one or more therapeutic agents. In some embodiments, one or more therapeutic agents may be selected from the group consisting of an AAV-based therapy or other gene delivery based therapy, messenger RNA or miRNA therapeutics, a pharmacological inhibitor, a growth factor, a gene therapy agent, a drug, a biological, a biomimetic, or synthetic therapy, or a combination thereof.

The present disclosure provides technologies for treating and/or preventing a neuropathic condition comprising administration of a provided therapeutic agent using systems and methods as described herein to a subject suffering from, susceptible to, and/or displaying symptoms of the neuropathic condition. In some embodiments, provided therapeutic agents for treatment of a neuropathic condition as described herein may be formulated for any route of administration described herein. In some embodiments, provided therapeutic agents may be formulated for topical administration. In some embodiments, provided therapeutic agents may be formulated for sub-dermal administration. In some embodiments, provided therapeutic agents may be formulated for transdermal administration. In some embodiments, provided therapeutic agents may be formulated for subcutaneous administration.

In some embodiments, such a provided treatment (e.g. therapeutic agents or stimulation) may be administered locally using systems as described herein to an affected site (e.g., axillae, hands, feet, scalp, face, neck, back, arms, chest, legs, etc., as appropriate to a particular neuropathic condition being treated). In some embodiments, local administration may be achieved by topical administration. In some embodiments, local administration may be achieved by sub-dermal administration. In some embodiments, local administration may be achieved by transdermal administration. In some embodiments, local administration may be achieved by subcutaneous administration.

### V. ADMINISTRATION SITES

The technologies of the present disclosure are suitable for both human and veterinary use. Subjects suffering from any neuropathic disorder, which would benefit from early onset detection of nerve degradation, body fluid collection and analyses, and/or treatment delivery using technologies disclosed herein.

Any site suitable for needle (e.g. microneedle) based detection and/or drug delivery is a suitable administration site. In some embodiments, an administration site is the skin overlying a muscle or muscle group of a subject. In some embodiments, administration site is hairless. In some embodiments, administration site is on the torso. In some embodiments, administration site is on the back. In some embodiments, administration site is on the chest. In some embodiments, administration site is on the head. In some embodiments, administration site is on the scalp. In some embodiments, administration site is on the face. In some embodiments, administration site is on the neck. In some embodiments, administration site is on the hands. In some embodiments, administration site is on the feet. In some embodiments site is on the arms. In some embodiments, administration site is on the legs.

In some embodiments, administration site is affected by a neuropathic condition. In some embodiments, administration site is the skin overlying a muscle or muscle group affected by a neuromuscular condition. In some embodiments, length of needles or microneedles used in systems described herein are adjusted based on skin thickness of administration site.

### VI. EXEMPLIFICATION

### Example 1: Measurement of Adipose Tissue Neuropathy in Human and Mouse Subcutaneous Tissue

The present Example demonstrates measurement of adipose tissue neuropathy in human and mouse subcutaneous adipose tissue. Specifically, the hypothesis that adipose tissue becomes neuropathic in conditions that include aging, diabetes, obesity, and certain diets was verified.

Protein levels were tested in the adipose tissue of (i) obese humans and mice and (ii) aging humans and mice, in accordance with previously known protocols.

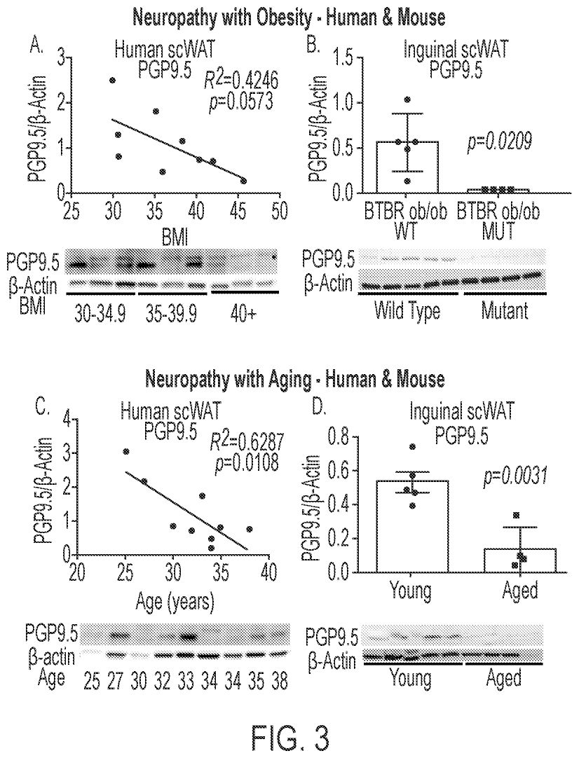

As shown in FIG. 3 measurement of protein levels of the pan-neuronal marker protein gene product (9.5) or PGFP 9.5 revealed a striking loss of innervation in subcutaneous white adipose tissue (scWAT) of humans (A,C) and mice (B, D) in both obesity/diabetes (top panels) and aging (bottom panels). The BTBR ob/ob mutant mouse (MUT) was used for diabetic peripheral neuropathy, as previously characterized in skin and hind paw by the Feldman Lab.

### Example 2: Detecting and Treating Peripheral Neuropathy in Subjects

The present Example lists the protocol for detection and/or treatment of peripheral neuropathy in subjects using the system described herein.

The device as described herein is connected to portable electronics, run by a software system.

Step 1: The area of skin to be assessed is determined. This will be a clinical determination based on extent of disease progression—early disease affects skin surface of feet and hands, later on the progression of nerve die back moves internally to deeper tissue layers, and up the legs and arms to the torso. Since skin depth varies depending on location on the body, the skin area being chosen determines the exact array fitted to the electronics—the size of the array to cover the skin surface, and the penetration depth of the needles.

Step 2: Once the array is chosen and attached to the electronics, the needles (e.g. microneedles) are placed on the patient's skin and inserted until the plastic backing is flush with the skin surface (providing the optimal penetration depth). From here, the associated electronics/software can begin recording nerve electrical activity, impedance or other indicators of nerve conductance, such as compound action potentials, from each needle in succession.

Step 3: If recordings require stimulation, electrical stimulation is applied first, followed by nerve recordings. If recordings do not require stimulation, then recordings begin immediately. Data is recorded from each microneedle in the attached software and stored in an FDA/HIPPA compliant manner. The software also contains an algorithm to assess and diagnose based on data recorded.

Step 4: If desired, the needles remain in place and may be used for either A) delivery of substances, such as medications (such as, biologics or drugs); or B) sampling of interstitial fluid.

Step 5: If required, additional treatments are applied including cold stimulation (via chemical or physical means or otherwise), or mechanical stimulation (such as vibration).

### Example 3: Measurement of Nerve Conductance and Delivery of Solubilized Product in Mice

The present Example demonstrates measurement of nerve conductance in mice and subdermal delivery of test treatment solution using the system described herein. Specifically, the hypothesis that nerve conductance is reduced in the skin and underlying tissue of diabetic mice is verified.

Nerve conductance measurements were obtained from mice using the protocol outlined in Example 2.

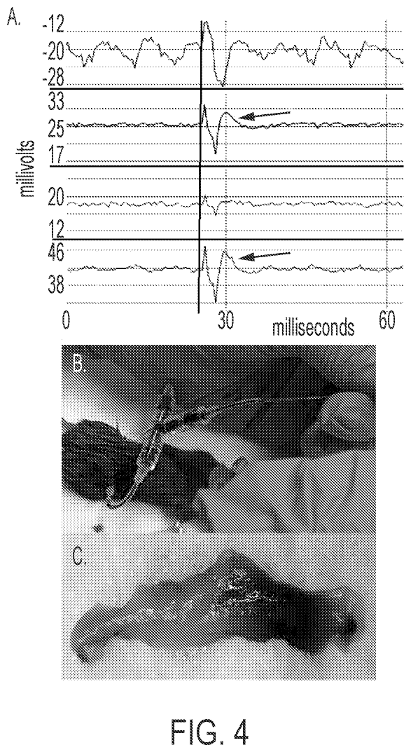

A needle array was used to record nerve conductance from mouse flank skin. Compound action potentials were recorded after 50 mV stimulation, shown in FIG. 4 as the vertical blue line. The arrow in FIG. 4A indicates recorded compound action potentials. Subsequently, Evan's Blue dye was subdermally injected through the needle array, as shown in FIG. 4B. FIG. 4C shows successful delivery of the injected dye solution in to the inguinal subcutaneous adipose tissue.

### Example 4: Peripheral Neuropathy Assessments for Dietary and Genetic Models of DPN

The present Example demonstrates PN assessments in dietary and genetic mouse models using the system described herein.

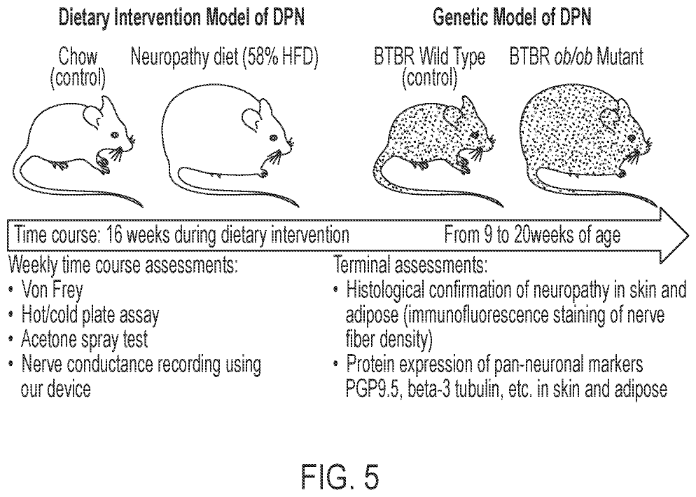

Concurrent electrophysiological measurements are performed in a cohort of diabetic neuropathy mice (N=6 BTBR ob/ob and N=6 wild-type controls, followed by N=6 chow and N=6 diet-induced DPN), as illustrated in FIG. 5 which are compared with other accepted assessments of DPN in mice including Von Frey, Acetone test, and Cold Plate test. These data are collected over the same time course of diabetes development from 9-20 weeks, with weekly measurements in paw and flank skin. At the end of 20 weeks animals are sacrificed and degree of DPN assessed through immunofluorescent staining of the skin for PGP9.5 and quantification of intraepidermal nerve fiber density.

For dietary intervention model, adult (9 week old) male mice are placed on either a chow or neuropathy diet (58% high fat diet (HFD)) for 16 weeks (which is the time necessary to develop DPN). Standard assessments of DPN (left bullet points in FIG. 5) are taken along with nerve conductance recordings (functional assessments) weekly for comparison. Terminal assessments (right bullet points of FIG. 5) will confirm DPN.

As anticipated, total nerve conductance recorded across the array, as well as nerve conductance at each individual needle is observed to reduce as animals progress to later stages of diabetes and neuropathy. In addition, loss of signal is observed at skin surface, then at deeper tissue layers, as indicated in the model figure.

### Example 5: Measurement of Nerve Conductance in Diabetic and Healthy Mice

The present Example demonstrates measurement of nerve conductance in obese/diabetic mice versus healthy mice using the system described herein. Specifically, the hypothesis that nerve conductance is reduced in the skin and underlying tissue of diabetic mice is verified.

Unless stated otherwise, materials and methods from Example 2 (e.g. for detecting and treating peripheral neuropathy) are performed as needed. For example, nerve conductance measurements are obtained from mice as outlined in Example 2.

BTBR ob/ob (Mutant/diabetic), and BTBR WT (healthy/control) male mice that are 12-16 weeks old are used. A cohort size of N=3-4 per group is used.

To anesthetize the mice, isoflurane 1-1.5% in pure O₂ is used. This experiment is performed utilizing the BioPac nerve conductance rig (e.g. HO₃ nerve conduction—MP36/35) that has been successfully tested on Limulus optic nerve with a hollow recording MN array (an array of 9 needles).

Nerve Recordings: Initial nerve conductance recordings from healthy mice, followed by diabetic mice are conducted at a depth necessary to measure nerve conductance in mouse, for example at a depth of 0.5 mm from the surface of the skin. Subsequent measurements in healthy and diabetic mice are taken at various depths ranging 1-1.5 mm, in order to reach underlying tissues, including subcutaneous adipose tissue. For each of these recordings, random electrical potentials are detected from any and all peripheral nerves innervating the tissue(s). Microvolt/millivolt averages are collected over a 10 minute period. If no differences in nerve conductance are detected, recordings may be extended beyond that time, or nerves may be stimulated by cold compress on skin.

As observed from the nerve conductance data collected, the nerve conductance recordings are lower in in diabetic tissues found in diabetic mice as compared to healthy tissue in healthy mice. Furthermore, the nerve conductance recordings are worse (i.e. lower) closer to the skin surface where the neuropathy begins.

Interstitial Fluid Sampling: In the same animals, a needle array (for example, a 9 hollow needle array) is connected to tubing and a syringe in order to apply negative pressure and collect interstitial fluid, at each of the needle depths used for recordings (0.5 mm, 1-1.5 mm).

The collected interstitial fluid sample is used for downstream testing and comparison of various biomarkers in diabetic mice versus healthy mice.

Delivery of Substances: In the same animals, the hollow recording needle (9-needle array) is used to deliver substances at the same depths at which the recordings were obtained (0.5 mm, 1-1.5 mm). These substances may include treatments that prevent or halt the progression of PN in diabetic mice. Furthermore, nerve recordings after delivery of treatment are obtained to verify PN regression, and/or treatment.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A method of evaluating and/or treating a subject for neuropathy, the method comprising:
placing a device comprising
a substrate; and
an array of needles disposed on or through the substrate;
wherein one or more of the needles is hollow through the entire length of the needle;
wherein the array is configured to allow fluid transport through at least one hollow needle of the array; and
wherein the array is configured to receive and communicate one or more electrical signals;
on or below a surface of the skin of the subject,
inserting the one or more needles into the skin and/or subcutaneous tissue of the subject, wherein at least one of the inserted needles is capable of transmitting electrical signals to and/or from the penetrated tissue; and
performing one or more of an electrical measurement, a stimulation, fluid sampling, and administration of one or more therapeutic agents using at least one of the one or more inserted needles, over a period of time to evaluate and/or treat a subject.

2. The method of claim 1, wherein the step of performing comprises performing two or more of an electrical measurement, a stimulation, fluid sampling, and administration of agents using at least one of the one or more inserted needles, over a period of time to evaluate and/or treat a subject.

3. The method of claim 1 or claim 2, wherein performing an electrical measurement comprises obtaining a measure of nerve conductance and/or tissue electrical activity, and/or local field potentials.

4. The method of any one of claims 1 to 3 further comprising:
determining, by comparing the performed electrical measurement with a similar electrical control measurement, a likelihood that the subject suffers from neuropathy.

5. The method of any one of claims 1 to 4, wherein the step of performing an electrical measurement over the period of time comprises:
measuring electrical potential (or current) via at least one of the inserted needles over the period of time, wherein the electrical potential (or current) is measured versus a reference electrode; and
determining a measure of nerve conductance associated with the measured electrical potential (or current).

6. The method of claim 5, wherein the measure of nerve conductance is determined based on a characteristic frequency of potential (or current) maxima measured in a given period of time.

7. The method of claim 5, wherein the measure of nerve conductance is determined based on a characteristic voltage associated with potential (or current) maxima measured in a given period of time.

8. The method of claim 5, wherein the measure of nerve conductance is determined based on a characteristic voltage level of the measured electrical potential (or current) in a given period of time.

9. The method of claim 5, wherein the measure of nerve conductance is determined based on an average of the measured electrical potentials (or currents) acquired from each inserted needle of the array.

10. The method of any one of claims 1 to 4, wherein performing an electrical measurement comprises:
at each of a plurality of target depths within the skin or subcutaneous tissue, measuring electrical potential (or current) of the at least one inserted needle over the period of time; and
determining, for each of the plurality of target depths, a measure of nerve conductance associated with the measured electrical potential (or current) for the target depth.

11. The method of claim 10, wherein the plurality of target depths comprise two or more depths within the skin and/or subcutaneous tissue associated with neuropathy.

12. The method of claim 10, wherein the measure of nerve conductance at each of the plurality of target depths comprises a measurement of nerve die-back as a function of distance from a skin surface inward.

13. The method of claim 12, wherein the measurement of nerve die-back provides a measure of delivery of one or more therapeutics to one or more of the plurality of target depths.

14. The method of claim 10, wherein the measure of nerve conductance is determined based on a characteristic frequency of potential (or current) maxima measured in a given period of time.

15. The method of claim 10, wherein the measure of nerve conductance is determined based on a characteristic voltage associated with potential (or current) maxima measured in a given period of time.

16. The method of claim 10, wherein the measure of nerve conductance is determined based on a characteristic voltage level of the measured electrical potential (or current) in a given period of time.

17. The method of claim 10, wherein the measure of nerve conductance is determined based on an average of the measured electrical potentials (or currents) acquired from each inserted needle of the array.

18. The method of any one of claim 5 or 10, wherein the step of determining the measure of nerve conductance comprises:
determining a characteristic frequency of potential (or current) maxima measured in a given period of time; and
comparing the characteristic frequency to a pre-determined threshold frequency to determine the measure of nerve conductance.

19. The method of any one of claim 5 or 10, wherein the step of determining the measure of nerve conductance comprises:
determining a characteristic value associated with potential (or current) maxima measured in a given period of time; and
comparing the characteristic value to a pre-determined threshold value in order to determine the measure of nerve conductance.

20. The method of claim 19, wherein the characteristic value is an average of one or more maxima of the measured electrical potential (or current) during the given period of time.

21. The method of any one of claim 5 or 10, wherein the step of determining the measure of nerve conductance comprises:
determining a characteristic voltage (or current) level of the measured electrical potential (or current) in a given period of time; and
comparing the characteristic voltage (or current) level to a pre-determined threshold value in order to determine the measure of nerve conductance.

22. The method of any one of claim 5 or 10, wherein the step of determining the measure of nerve conductance comprises:
determining a characteristic amplitude of the measured electrical potential (or current) in a given period of time; and
comparing the characteristic amplitude to a pre-determined threshold value in order to determine the measure of nerve conductance.

23. The method of any one of claim 5 or 10, wherein the step of determining the measure of nerve conductance comprises:
determining a characteristic pattern of firing of neurons at the measured electrical potential (or current) in a given period of time; and
comparing the characteristic pattern of firing of neurons to a pre-determined threshold pattern in order to determine the measure of nerve conductance.

24. The method of any one of claims 1-23, wherein the step of performing comprises:
sampling interstitial fluid of the subject from the skin and/or subcutaneous tissue using the one or more inserted needles;
determining, based at least in part on properties of the sampled interstitial fluid, the status of disease progression in the subject.

25. The method of any one of claims 1-24, wherein administering the one or more therapeutic agents to the subject reverses and/or prevents progression of disease.

26. The method of claim 25, wherein the one or more therapeutic agents is selected from the group consisting of a pharmacological inhibitor, a growth factor, a gene therapy agent, a drug, a biological, or a combination thereof.

27. The method of claim 25, wherein administering the one or more therapeutic agents to the subject comprises providing the one or more therapeutic agents to the skin and/or subcutaneous tissue of the subject.

28. The method of any one of claims 1-27, wherein the stimulation is achieved by electrically stimulating the skin or subcutaneous tissue of the subject to prevent and/or reverse progression of disease.

29. The method of any one of claims 1-28, wherein the stimulation through temperature regulation comprises cooling the skin and/or subcutaneous tissue of the subject.

30. The method of claim 29, wherein the cooling is performed using ice, a thermoelectric cooler, or the like.

31. The method of any one of claims 1-29, wherein the stimulation is achieved by mechanically stimulating the skin and/or subcutaneous tissue of the subject.

32. The method of claim 31, wherein the stimulating is performed using a means to provide mechanical vibration thereby mechanically stimulating muscle and/or other tissues near the one or more inserted needles to stimulate nerve re-growth.

33. The method of any one of the preceding claims, wherein the electrical control measurement is obtained from healthy control subjects, a patient data sample taken earlier in disease progression, or from a healthy region of tissue on the subject's body.

34. The method of claim 18, wherein the pre-determined threshold frequency is obtained from a healthy subject.

35. The method of any one of claim 19, 21, or 22, wherein the pre-determined threshold value is obtained from a healthy subject.

36. The method of claim 23, wherein the pre-determined threshold pattern is obtained from a healthy subject.

37. A system for evaluating, diagnosing, preventing, and/or treating neuropathy in a subject, comprising:
a device, wherein the device comprises:
a substrate; and
an array of needles disposed on or through the substrate;
wherein one or more of the needles is hollow through the entire length of the needle;
wherein the array is configured to allow fluid transport through at least one hollow needle of the array; and
wherein the array is configured to receive and communicate one or more electrical signals;
a means for providing fluid flow, wherein means for providing fluid flow is fluidically coupled to at least one hollow needle of the device; and
one or more digital processors and associated electronics configured to receive data from the device and to control the device.

38. The system of claim 37, further comprising a battery to power the system.

39. The system of claim 37, further comprising a wireless transmission module to wirelessly exchange data to and from an operating device.

40. The system of claim 37, further comprising a PC-card and/or an amplifier.

41. The system of claim 37, wherein the processor is configured to perform a method of any one of method claims 1-31.

42. The system of claim 37, wherein the means for providing fluid flow is or comprises a mechanical pump and/or a syringe pump.

43. The system of any one of claims 37 to 42, wherein the processor is configured to, responsive to a determination by the processor that the subject suffers from neuropathy, automatically provide a fluid to the skin and/or subcutaneous tissue of the subject via a hollow needle of the device, wherein the fluid comprises one or more therapeutic agents.

44. A device comprising:
a substrate; and
an array of needles disposed on or through the substrate;
wherein one or more of the need [symbol] hrough the entire length of the needle;
wherein the array is configured to allow fluid transport through at least one hollow needle of the array; and
wherein the array is configured to receive and communicate one or more electrical signals.

45. The device of claim 44, wherein one or more needles in the array is or comprises stainless steel, silicon, platinum, gold, silver, copper, any electrically conductive metal, or any combination thereof.

46. The device of any one of claim 44 or 45, wherein one or more needles in the array further comprises a first coating to penetrate skin to epidermis, dermis, or further beneath the skin (e.g., such as to adipose or muscle tissues).

47. The device of any one of claims 44 to 46, wherein one or more needles in the array further comprises a second coating to insulate an electrical signal.

48. The device of any one of claims 44 to 47, comprising two or more coatings.

49. The device of claim any one of claims 44 to 48, wherein the one or more needles in the array is coated with an electrically conductive material.

50. The device of any one of claims 44 to 49, wherein one or more needles in the array is at least partially filled with an electrically conductive material.

51. The device of any one of claim 49 or 50, wherein the electrically conductive material is selected from the group consisting of stainless steel, silicon, platinum, gold, silver, copper, polymers, any metal, or any combination thereof.

52. The device of any one of claims 44 to 51, wherein the fluid transport may be bidirectional.

53. The device of claim 52, wherein the fluid transport is between the device and one or more of skin, interstitial fluid, and/or subcutaneous tissue of a subject.

54. The device of any one of claims 44 to 53, wherein one or more of the needles is a stimulating electrode.

55. The device of any one of claims 44 to 54, wherein one or more needles is a reference electrode or ground electrode.

56. The device of any one of claims 44 to 55, wherein the one or more needles comprise an electrically conductive wire through the hollow opening.

57. The device of any one of claims 44 to 56, wherein the needles are microneedles.

58. The device of any one of claims 44 to 57, wherein one or more needles in the array is or comprises an electrically non-conductive material and wherein the one or more needles is at least partially filled or coated with an electrically conductive material.

59. The device of any one of claims 44 to 58, wherein the length of one or more needles is configured to allow electrical measurement in and therapeutic delivery to a target depth within the skin and/or subcutaneous tissue of a subject.

60. The device of claim 59, wherein at least two or more needles are of different length.

61. The device of any one or claim 59 or 60, wherein the length of the needles between 10 μm to 12 mm.

62. The device of claim 59, wherein an outer diameter of the needles is 100 μm or greater.

63. The device of claim 59, wherein an inner diameter of the needles is 10 μm or greater.

64. The device of any one of claims 44 to 63, wherein the substrate and/or array are configured to allow adjustment of the depth of penetration of one or more needles into skin and/or subcutaneous tissue of a subject, thereby allowing measurement and/or treatment at a plurality of target depths within the skin, and/or subcutaneous tissue of the subject.

65. The device of claim 64, wherein the depth of penetration of one or more needles is configured using an adjustable spacer.

66. The device of any one of claims 44 to 65, further comprising a cooling mechanism.

67. The device of any one of claims 44 to 66, further comprising a mechanism of stimulation.

68. The device of claim 67, wherein the mechanism of stimulation is electrical and wherein electrical stimulation is achieved by administration of approximately 20% greater voltage/current than required for maximal stimulus.

69. The device of claim 67, wherein the mechanism of stimulation is mechanical.

70. The device of claim 69, wherein the mechanical stimulation is achieved by vibration.

71. The device of claim 67, wherein the mechanism of stimulation is through temperature regulation.

72. The device of any one of claims 44 to 71, wherein the substrate is or comprises a flexible backing.

73. The device of any one of claims 44 to 72, wherein the device is 3D printed or otherwise fabricated.

74. The device of any one of claims 44 to 73, wherein the substrate is a square, rectangle, or other shape suitable to accommodate curvature of various body parts.

75. The device of any one of claims 44 to 74, wherein the substrate has a surface area of at least 1 square inch.

76. The device of claim 72, wherein the substrate has at least one dimension of at most 8 inches.

77. The method of claim 5, wherein the measure of nerve conductance is determined based on a characteristic amplitude of current measured in a given period of time.

78. The method of claim 10, wherein the measure of nerve conductance is determined based on a characteristic amplitude of current measured in a given period of time.

79. The method of any one of claim 5 or 10, wherein the step of determining the measure of nerve conductance comprises:
determining a characteristic amplitude of current measured in a given period of time; and
comparing the characteristic amplitude to a pre-determined threshold amplitude to determine the measure of nerve conductance.

80. The method of any one of claims 5 to 10, wherein the measure of nerve conductance is determined based on the measure of local field potentials.

81. The method of any one of claims 5 to 10, wherein the measure of nerve conductance is determined based on frequency of current pulses (e.g. electrical spikes (e.g. current spikes)).

82. A method of evaluating nerve health, nerve function, and/or nerve regrowth in a subject, the method comprising:
placing a device comprising
a substrate; and
an array of needles disposed on or through the substrate;
wherein one or more of the needles is hollow through the entire length of the needle;
wherein the array is configured to allow fluid transport through at least one hollow needle of the array; and
wherein the array is configured to receive and communicate one or more electrical signals;
on or below a surface of the skin of the subject,
inserting the one or more needles into the skin and/or subcutaneous tissue of the subject, wherein at least one of the inserted needles is capable of transmitting electrical signals to and/or from the penetrated tissue; and
performing one or more of an electrical measurement, a stimulation, fluid sampling, and administration of one or more agents using at least one of the one or more inserted needles, over a period of time to evaluate and/or treat a subject.
